SEQUENCE LISTING (8 sequences)

SEQ ID NO: 1 - protein, 107 aa, synthetic construct
  REGION 1..107: note = source = /note="Description of Artificial Sequence: Synthetic polypeptide"
  REGION 1..107: note = source = /note="LIGHT CHAIN VARIABLE REGION OF CLONE CL-2C"
EIVMTQSPATLSVSPGERATLSCRASQSIGTNIHWYQQKPGQAPRLLIYYASESISGIPARFSGSGSGTEFTLTISSLQSEDFAVYYCQQSWSWPTTFGGGTKVEIK

SEQ ID NO: 2 - protein, 123 aa, synthetic construct
  REGION 1..123: note = source = /note="Description of Artificial Sequence: Synthetic polypeptide"
  REGION 1..123: note = source = /note="HEAVY CHAIN VARIABLE REGION OF CLONE CL-2C"
QVQLVQSGAEVMKPGSSVKVSCKASGYTFSWYWLEWVRQAPGHGLEWMGEIDPGTFTTNYNEKFKARVTFTADTSTSTAYMELSSLRSEDTAVYYCARFSHFSGSNYDYFDYWGQGTLVTVSS

SEQ ID NO: 3 - protein, 11 aa, synthetic construct
  REGION 1..11: note = source = /note="Description of Artificial Sequence: Synthetic peptide"
  REGION 1..11: note = source = /note="TES-C21 CDRL1 SEQUENCE (TABLE 1)"
RASQSIGTNIH

SEQ ID NO: 4 - protein, 7 aa, synthetic construct
  REGION 1..7: note = source = /note="Description of Artificial Sequence: Synthetic peptide"
  REGION 1..7: note = source = /note="TES-C21 CDRL2 SEQUENCE (TABLE 1)"
YASESIS

SEQ ID NO: 5 - protein, 9 aa, synthetic construct
  REGION 1..9: note = source = /note="Description of Artificial Sequence: Synthetic peptide"
  REGION 1..9: note = source = /note="CDRL3 VARIANT SEQUENCE #2 (TABLE 1)"
QQSWSWPTT

SEQ ID NO: 6 - protein, 5 aa, synthetic construct
  REGION 1..5: note = source = /note="Description of Artificial Sequence: Synthetic peptide"
  REGION 1..5: note = source = /note="CDRH1 VARIANT SEQUENCE #1 (TABLE 1)"
WYWLE

SEQ ID NO: 7 - protein, 17 aa, synthetic construct
  REGION 1..17: note = source = /note="Description of Artificial Sequence: Synthetic peptide"
  REGION 1..17: note = source = /note="CDRH2 VARIANT SEQUENCE #2 (TABLE 1)"
EIDPGTFTTNYNEKFKA

SEQ ID NO: 8 - protein, 14 aa, synthetic construct
  REGION 1..14: note = source = /note="Description of Artificial Sequence: Synthetic peptide"
  REGION 1..14: note = source = /note="TES-C21 CDRH3 (TABLE 1)"
FSHFSGSNYDYFDY

(19) United States
(12) Patent Application Publication
(10) Pub. No.: US 2025/0320311 A1
(43) Pub. Date: Oct. 16, 2025

(54) TREATMENT OF ALLERGIC REACTIONS USING ANTI-IGE ANTIBODIES

(71) Applicant: NOVARTIS AG, Basel (CH)

(72) Inventors: Ivan BOTTOLI, Zug (CH); Maciej CABANSKI, Allschwil (CH); Anna EGGER, Baselland (CH); Aurelie GAUTIER, Huningue (FR); Fernando Takeshi KAWAKAMI, Bottmingen (CH)

(21) Appl. No.: 18/718,941

(22) PCT Filed: Dec. 12, 2022
(86) PCT No.: PCT/IB2022/062066

§ 371 Date: Jun. 12, 2024

Related U.S. Application Data
(60) Provisional application No. 63/289,373, filed on Dec. 14, 2021.

(51) Int. Cl. C07K 16/42; A61K 39/00; A61P 37/08

(52) U.S. Cl. CPC C07K 16/4291; A61P 37/08; A61K 2039/545; C07K 2317/76

(57) ABSTRACT

The present disclosure relates to methods for modifying the course of a disease or disorder involving IgE, in particular an IgE mediated allergic reactions to one or more allergens, in subjects having such disease or condition.

### TECHNICAL FIELD

The present disclosure relates to treatments and methods of preventing or treating diseases or disorders in subjects in need of treatment, e.g. preventing or treating food allergy to one or more allergens (e.g. food allergy, peanuts allergy, milk allergy or egg allergy), or anaphylaxis, using an anti-IgE antibody e.g. ligelizumab.

### BACKGROUND OF THE DISCLOSURE

Immunoglobulin E (IgE) is an antibody associated with hypersensitivity and allergic reactions. IgE mainly binds on the high-affinity IgE receptor (FcεRI) on mast cells, basophils and dendritic cells and hence decreases the induction of the regulatory T cells.

Food allergy affects millions of people of all ages in all nations, and a rapidly rising prevalence suggests it is an emerging population health priority (Warren et al 2020). The underlying pathogenesis of food allergy involves an immunologic mechanism in which allergen-specific IgE is synthesized in response to allergen exposure and binds to high affinity receptors for IgE (FcεRI receptors) via its Fc region on the surface membranes of mast cells and basophils. (Sampson et al 2006). Food allergies affect almost 10% of people worldwide, with consistent epidemiology trends observed across North America, Europe, Asia and Australia; the most common allergens are: peanut, tree nuts, seafood, egg, milk, wheat, soy and seeds (Warren et al 2020, Sicherer and Sampson 2017). Allergy to peanut, tree nuts and seafood are usually lifelong (Jones and Burks 2017, Sicherer and Sampson 2017). Additionally, between one-third to one-half of food allergic patients—including adults—are likely to be allergic to more than one food (Gupta et al 2011, Gupta et al 2019).

A food allergy is an adverse immune response to a food allergen, e.g., a food protein. Common food allergens are found in shellfish, peanuts, tree nuts, fish, milk, eggs, soy and fresh fruits such as strawberries, mango, banana, and apple. Immunoglobulin-E (IgE)-mediated food allergies are classified as type-I immediate hypersensitivity reactions. These allergic reactions have an acute onset (from seconds to one hour) and the accompanying symptoms may include angioedema (soft tissue swelling of the eyelids, face, lips, tongue, larynx and trachea); hives; itching of the mouth, throat, eyes, skin; gastrointestinal symptoms such as nausea, vomiting, diarrhea, stomach cramps, or abdominal pain; rhinorrhea or nasal congestion; wheezing, shortness of breath, or difficulty swallowing; and even anaphylaxis, a severe, whole-body allergic reaction that can result in death. Furthermore, anaphylaxis is a potentially life-threatening condition.

Currently the standard of care for food allergy is limited to strict avoidance of the inciting food(s), rescue medication in case of unintentional exposure, and community wide interventions for schools (i.e., peanut free classrooms) and restaurants (i.e., ingredient alerts). Recently, a peanut oral immunotherapy was approved to mitigate allergic reactions during accidental exposure to peanuts. Yet this treatment is not fundamentally changing the unmet medical need in this space as it is only targeting one allergen; is indicated only for a subset of age groups and might not be suitable for all peanut allergic patients.

Allergen-specific oral immunotherapy (OIT) consists of daily administration of small, gradually increasing amounts of allergen to induce allergen desensitization.

Following the identification of IgE as a principal player in allergic diseases and the advent of monoclonal antibody technology in the 1970s, monoclonal antibodies to IgE were developed to the site on IgE that bound the FcεRI receptor (Baniyash et al 1988). The monoclonal anti-IgE treatment with TNX-901 was shown to be able to significantly increase the threshold of sensitivity to peanut antigen, as assessed by oral food challenge, in a dose dependent manner, to levels that should translate into at least partial protection against most unintended ingestions of peanut (Leung et al 2003).

Xolair® (omalizumab) is a recombinant DNA-derived humanized monoclonal antibody that selectively binds to free, circulating human immunoglobulin E (IgE) thus inhibiting IgE binding to IgE receptors on the surface of mast cells and basophils resulting in decreased release of allergic mediators. By binding to free, circulating IgE, omalizumab also lowers serum free IgE levels and down-regulates the number of IgE receptors on the surface of mast cells and basophils. Omalizumab is widely used for the treatment of Allergic asthma, allergic rhinitis and chronic spontaneous urticaria (CSU). Omalizumab facilitated rapid peanut desensitization in highly allergic patients. In another trial, evaluating whether omalizumab facilitated desensitization in patient with multi-food allergies, including 48 patients with allergy to 2-5 foods, after completing 8 weeks of omalizumab monotherapy, treated patients tolerated a median dose of mixed food of 2380 mg (max dose 2380 mg) compared to 55 mg in the placebo group. Although well tolerated, omalizumab administration can induce side effects ranging from skin inflammation (at the injection site) to systemic anaphylaxis (in 0.1-0.2% of patients).

Due to rising prevalence (including allergy to multiple food allergens), currently limited therapeutic options, potentially life-threatening consequences, the inability of food avoidance alone to offer protection and lifelong disease burden in many, there is a significant medical need to develop safe and efficacious therapies for food allergy suitable for allergic patients. The standard of care upon accidental exposure to allergens consists of allergen avoidance and epinephrine administration. The unmet need, in particular for therapeutic solutions not requiring regular allergen administration, is even more significant in children and adolescents.

Furthermore, anaphylaxis is the most severe clinical presentation of acute systemic allergic reactions. Manifestations include cutaneous, respiratory, cardiovascular, or gastrointestinal signs and symptoms. Although anaphylaxis is frequently attributed to exposure to specific foods, drugs, and insect venoms in sensitive individuals, a causative factor is not identified in 30% to 50% of patients with recurrent anaphylactic episodes (idiopathic anaphylaxis). Current therapeutic options for the treatment of anaphylaxis, e.g., idiopathic anaphylaxis are limited with variable efficacy. Anaphylaxis has a global incidence between 50 and 112 episodes per 100 000 person-years. The occurrence of anaphylaxis has increased in recent years, and subsequently, there is a need to provide for therapeutic solutions suitable for subjects in need thereof.

Ligelizumab is a humanized monoclonal antibody with higher affinity binding to human immunoglobulin E (IgE) than omalizumab. Upon binding, ligelizumab is able to block the interaction of IgE with both the high and low affinity IgE receptors (FcεRI and FcεRII). When subjects receive ligelizumab, circulating IgE is rapidly bound by the anti-IgE antibody and becomes inaccessible to IgE receptors on mast cells and basophils. IgE is necessary for the enhanced expression of the FcεRI seen in atopic subjects, and a decrease in FcεRI expression on circulating basophils accompanies ligelizumab treatment. Other potentially beneficial effects from anti-IgE therapy using ligelizumab include decreased IgE production, reduced B cell numbers and reduced cytokine production by T cells. This mechanism confers already benefit to subjects with chronic spontaneous urticaria (CSU) by preventing the itchy hives and angioedema that are associated with degranulation (histamine release) of mast cells and basophils.

### SUMMARY OF THE DISCLOSURE

We identified that high level of suppression of free IgE, reduction in basophil FcεRI expression and thus basophil surface IgE elicited anti-IgE antibody, such as ligelizumab should result in a more efficient desensitization against the allergen; therefore, ensuring protection against allergic reactions, e.g. food allergic reaction, or anaphylaxis, by decreasing allergen sensitivity to allergens as a therapeutic solution irrespective of the triggering factor. Ligelizumab demonstrated dose- and time-dependent suppression of free IgE, basophil FcεRI, basophil surface IgE, and skin prick test responses to allergen, superior in extent and duration to those observed with omalizumab. Ligelizumab binds with higher affinity to human IgE than omalizumab. Furthermore, superior affinity and pharmacodynamic (PD) outcomes of ligelizumab compared to omalizumab may translate into superior posology and superior clinical efficacy in the prevention or treatment of Ig-E mediated allergic reaction, e.g., in the prevention or treatment of anaphylaxis.

Accordingly, disclosed herein are methods of preventing or treating food allergy, e.g., IgE mediated food allergy, to one or more allergens (e.g., food allergy, peanuts allergy, milk allergy or egg allergy), comprising administering a therapeutically effective amount of an anti-IgE antibody, e.g., an anti-IgE antibody that selectively binds to free, circulating human IgE (e.g., ligelizumab), to a subject in need thereof.

Accordingly, disclosed herein are methods of treating, preventing, or reducing anaphylaxis, e.g., IgE mediated anaphylaxis, comprising administering a therapeutically effective amount of an anti-IgE antibody, e.g., an anti-IgE antibody that selectively binds to free, circulating human IgE (e.g., ligelizumab), to a subject in need thereof.

In one embodiment, an anti-IgE antibody is provided having variable light chain region comprising CDRL1, CDRL2, and CDRL3 and a variable heavy chain region comprising CDRH1, CDRH2, and CDRH3, wherein CDRL1 consists of SEQ ID NO:3, CDRL2 consists of SEQ ID NO:4, CDRL3 consists of SEQ ID NO:5, CDRH1 consists of SEQ ID NO:6, CDRH2 consists of SEQ ID NO:7, and CDRH3 consists of SEQ ID NO:8, wherein the antibody binds specifically to IgE, and wherein said antibody is to be administered to a subject in need thereof, as a dose of from about 240 mg to about 600 mg.

In a preferred embodiment, an anti-IgE antibody designated QGE031 (ligelizumab) is provided. Specifically, QGE031 (ligelizumab) comprises variable light chain region comprising CDRL1, CDRL2, and CDRL3 and a variable heavy chain region comprising CDRH1, CDRH2, and CDRH3, wherein CDRL1 consists of SEQ ID NO:3, CDRL2 consists of SEQ ID NO:4, CDRL3 consists of SEQ ID NO:5, CDRH1 consists of SEQ ID NO:6, CDRH2 consists of SEQ ID NO:7, and CDRH3 consists of SEQ ID NO:8. The anti-IgE antibody ligelizumab comprises an immunoglobulin VH domain comprising the amino acid sequence set forth as SEQ ID NO:2 and an immunoglobulin VL domain comprising the amino acid sequence set forth as SEQ ID NO:1. Said antibody is to be administered to a subject in need thereof, as a dose of from about 300 mg to about 360 mg.

In one embodiment, the route of administration is subcutaneous or intravenous of the antibody according to the embodiments herein described, or a combination of subcutaneous or intravenous.

Some patients may benefit from a loading regimen (e.g., weekly for several weeks [e.g., 1 to 5 weeks, e.g., dosing at weeks 0, 1, 2, 3 and/or 4] or biweekly for several weeks (e.g., 2 to 8 weeks, e.g., dosing at weeks 0, 2, 4, and/or 6) followed by maintenance regimen, e.g. a monthly maintenance regimen. For example, an appropriate regimen for anti-IgE antibody can be weekly or bi-weekly for several weeks [e.g., 1 to 5 weeks, e.g., dosing at weeks 0, 1, 2, 3 and/or 4] followed by a monthly maintenance regimen.

In another example, an appropriate regimen for ligelizumab is a monthly regimen.

In some embodiments, the anti-IgE antibody, such as ligelizumab, may be administered to the subject at an initial dose of 240 mg delivered s.c., and the dose may be then adjusted if needed, as determined by a physician, based on subject IgE levels and body weight.

In some embodiments, the anti-IgE antibody, such as ligelizumab, may be administered to the subject at an initial dose of 300 mg delivered s.c., and the dose may be then adjusted if needed, as determined by a physician, based on subject IgE levels and body weight.

In yet another specific embodiment, a dose which comprises three unit doses of 120 mg ligelizumab is administered s.c. every four (4) weeks (q4w).

Ligelizumab may be administered quarterly, monthly, weekly or biweekly e.g. subcutaneously at a dosing of about 240 mg to 600 mg, e.g. about 280 mg to about 360 mg, e.g. about 300 mg to about 360 mg, or a e.g. about 300 mg, being administered, by subcutaneous injection, at an unit dose of about 120 mg, about 150 mg, about 240 mg, or about 300 mg.

Ligelizumab may be administered by subcutaneous (s.c.) injection, bi-weekly, or monthly at a dose of about 120 mg to about 240 mg, preferably about 240 mg.

Ligelizumab may be administered by subcutaneous (s.c.) injection, bi-weekly, or monthly at a dose of about 300 mg to about 360 mg, preferably about 300 mg or about 360 mg.

As herein defined, “unit dose” of ligelizumab refers to a s.c. dose that can be comprised between about 24 mg to 600 mg, e.g. about 72 mg to about 30 mg, e.g. about 100 mg to about 300 mg, or a e.g. about 120 mg to about 240 mg. For example, an unit s.c. dose is about 24 mg, about 72 mg, about 120 mg, about 150 mg, about 240 mg, or about 360 mg.

In one embodiment, the present invention comprises administering ligelizumab to a subject with food allergy, in the range of from about 24 mg to about 600 mg per treatment, preferably in the range of from about 72 mg to about 360 mg, preferably in the range of 100 mg to 360 mg, preferably of about 240 mg to about 360 mg per treatment. In one embodiment a patient receives from about 120 mg to about 240 mg per treatment. In one embodiment patient receives about 120 mg per treatment. In one embodiment patient receives about 240 mg per treatment. In one embodiment patient receives about 300 mg per treatment. In one embodiment patient receives about 360 mg per treatment In one embodiment patient receives about 20 mg, about 30 mg, about 60 mg, about 90 mg, about 120 mg, about 150 mg, about 180 mg, about 200 mg, about 210 mg, 240 mg, about 275 mg, or about 360 mg per treatment. In one embodiment the patient with food allergy, receives each treatment every 2 weeks, every 3 weeks, monthly (every 4 weeks), every 6 weeks, bimonthly (every 2 months), every 9 weeks or quarterly (every 3 months). In one embodiment the patient receives each treatment every 3 weeks. In one embodiment the patient receives each treatment every 4 weeks.

In one embodiment, the present invention comprises administering ligelizumab to a food-allergic subject, in the range of from about 24 mg to about 600 mg per treatment, preferably in the range of from about 72 mg to about 360 mg, preferably in the range of 120 mg to 360 mg, preferably of about 240 mg to about 360 mg per treatment, to prevent signs and symptoms of anaphylaxis during an oral food challenge. In one embodiment a subject receives from about 120 mg to about 360 mg per treatment In one embodiment subject receives about 120 mg per treatment. In one embodiment subject receives about 240 mg per treatment. In one embodiment subject receives about 300 mg per treatment. In one embodiment subject receives about 360 mg per treatment. In one embodiment subject receives about 20 mg, about 30 mg, about 60 mg, about 90 mg, about 120 mg, about 150 mg, about 180 mg, about 200 mg, about 210 mg, 240 mg, about 280 mg, about 300 mg, about 320 mg, or about 360 mg per treatment. In one embodiment the subject in need of anaphylaxis treatment, prevention or reduction, e.g. anaphylaxis during an oral food challenge, receives each treatment every 2 weeks, every 3 weeks, monthly (every 4 weeks), every 6 weeks, bimonthly (every 2 months), every 9 weeks or quarterly (every 3 months). In one embodiment the subject receives each treatment every 3 weeks. In one embodiment the subject receives each treatment every 4 weeks.

In one embodiment, the present invention comprises administering ligelizumab to a subject for the treatment, prevention, or reduction of IgE-mediated anaphylaxis, e.g. IgE-mediated idiopathic anaphylaxis, in the range of from about 24 mg to about 600 mg, preferably in the range of from about 72 mg to about 360 mg, preferably in the range of 120 mg to 360 mg, preferably of about 240 mg to about 360 mg. In one embodiment a subject receives from about 120 mg to about 360 mg per administration. In one embodiment subject receives about 120 mg. In one embodiment subject receives about 240 mg. In one embodiment subject receives about 300 mg. In one embodiment subject receives about 360 mg. In one embodiment subject receives about 300 mg. In one embodiment subject receives about 480 mg. In one embodiment subject receives about 20 mg, about 30 mg, about 60 mg, about 90 mg, about 120 mg, about 150 mg, about 180 mg, about 200 mg, about 210 mg, 240 mg, about 280 mg, about 300 mg, about 320 mg, or about 360 mg per treatment or preventive intervention. In one embodiment the subject in need of anaphylaxis treatment or prevention, receives each treatment or preventive intervention every 2 weeks, every 3 weeks, monthly (every 4 weeks), every 6 weeks, bimonthly (every 2 months), every 9 weeks or quarterly (every 3 months). In one embodiment the subject receives each treatment or preventive intervention every 3 weeks. In one embodiment the subject receives each treatment or preventive intervention every 4 weeks.

When safety concern raises, the dose can be down-titrated, preferably by increasing the dosing interval, preferably by doubling or tripling the dosing interval. For example 240 mg monthly or every 3 weeks regimen can be doubled to every 2 month or every 6 weeks respectively or tripled to every 3 month or every 9 weeks respectively.

In some embodiments, the anti-IgE antibody or binding fragment thereof is to be administered in combination with one or more additional agents. In some embodiments, the one or more additional agents is an agent for treatment of food allergy, e.g. OIT.

In some embodiments, the anti-IgE antibody or binding fragment thereof, e.g., ligelizumab, is to be administered as an adjunct to one or more agents for treatment of food allergy, e.g. OIT. Ligelizumab can be administered as an adjunct to OIT in methods for preventing food allergy reactions to any food allergens, e.g. severe food allergy to any food allergens.

In some embodiments, the anti-IgE antibody or binding fragment thereof, e.g. ligelizumab, is to be administered as adjunct to OIT, wherein the administration of the anti-IgE antibody or binding fragment thereof, e.g. ligelizumab, facilitates OIT by reducing the frequency and/or severity of adverse events associated with OIT, In some embodiments, the anti-IgE antibody or binding fragment thereof, e.g. ligelizumab, is to be administered as adjunct to OIT, wherein the administration of the anti-IgE antibody or binding fragment thereof, e.g. ligelizumab, facilitates OIT by shortening or eliminating the up-titration phase of the OIT.

In some embodiments, the anti-IgE antibody or binding fragment thereof, e.g. ligelizumab, is to be administered as adjunct to OIT, wherein the administration of the anti-IgE antibody or binding fragment thereof, e.g. ligelizumab, enables more subjects to reach OIT maintenance dose, when ligelizumab is administered as adjunct to OIT compared to OIT monotherapy administration.

In some embodiments, the anti-IgE antibody or binding fragment thereof, e.g. ligelizumab, is to be administered in combination with OIT, wherein the administration of the anti-IgE antibody or binding fragment thereof, e.g. ligelizumab, induces sustained unresponsiveness or immuno-tolerance to food allergen in subjects with IgE-mediated food allergy. For example, ligelizumab is administered 8 weeks prior to start of OIT, followed by a concomitant use of ligelizumab with OIT during up titration phase of OIT, and during the OIT maintenance phase.

In another aspect, the disclosure provides new dosing regimens for anti-IgE antibodies (e.g., ligelizumab) and binding fragments thereof that can be used in methods of treating or preventing food allergy.

In another aspect, the disclosure provides new dosing regimens for ligelizumab that can be used in methods of treating or preventing food allergy.

In another aspect, the disclosure provides new dosing regimens for ligelizumab that can be used in methods of treating or preventing prevent allergic reactions, including anaphylaxis, following accidental exposure to food allergens in conjunction with diet avoiding foods to which a subject is allergic.

Ligelizumab may be administered as monotherapy, e.g., when used in methods of treating or preventing food allergic reactions, when used in methods of preventing anaphylaxis following accidental exposure to food allergens.

In another aspect, the disclosure provides new dosing regimens for ligelizumab that can be used in methods of treating or preventing anaphylaxis from accidental exposure to one or more food allergens in a subject.

In another aspect, the disclosure provides new dosing regimens for ligelizumab that can be used in methods of treating, preventing or reducing anaphylaxis.

In another aspect, the disclosure provides new dosing regimens for anti-IgE antibodies (e.g., ligelizumab) and binding fragments thereof that can be used in methods of treating, preventing or reducing IgE-mediated allergic reaction in a subject with recurrent spontaneous anaphylaxis due to unknown and/or unavoidable triggers.

In another aspect, the disclosure provides new dosing regimens for anti-IgE antibodies (e.g., ligelizumab) and binding fragments thereof that can be used in methods of treating, preventing or reducing severe or serious allergic IgE-mediated reactions triggered by at least one allergen (e.g., insect stings/bites, venom, medications, e.g. beta-lactam antibiotics, NSAIDS, biological agents; aeroallergens, occupational allergens, radiocontrast media, natural rubber latex, seminal fluid).

In another aspect, the disclosure provides new dosing regimens for anti-IgE antibodies (e.g., ligelizumab) and binding fragments thereof that can be used in methods of treating, preventing or reducing severe or serious allergic IgE-mediated reactions of unidentified triggers (e.g., idiopathic anaphylaxis).

In some embodiments, the anti-IgE antibody, such as ligelizumab, may be administered to the subject at an initial dose of 300 mg delivered s.c., and the dose may be then adjusted if needed, as determined by a physician, based on subject IgE levels and body weight, e.g. based on subject IgE levels which can be total IgE level <350 IU/mL; or total IgE level 2 350 IU/mL.

In yet another specific embodiment, a dose which comprises two unit doses of 150 mg ligelizumab is administered s.c. every four (4) weeks (q4w).

Ligelizumab may be administered by subcutaneous injection, bi-weekly, or monthly at a dose of about 120 mg to about 480 mg, preferably about 360 mg or about 480 mg.

In some embodiments, the anti-IgE antibody, such as ligelizumab, may refer to antibodies which have demonstrated to be biosimilar to or interchangeable to ligelizumab. Those antibodies may be administered according the embodiments which refer to ligelizumab administration, as herein disclosed.

### DETAILED DESCRIPTION OF THE DISCLOSURE

### Embodiments

A1. An anti-IgE antibody or antigen binding fragment thereof for use in treating or preventing food allergy to one or more allergens in a subject in need thereof.
A2. An anti-IgE antibody or antigen binding fragment thereof for use in treating or preventing IgE mediated food allergy to one or more allergens in a subject in need thereof.
A3. The anti-IgE antibody or antigen binding fragment thereof according to embodiment A1, wherein the food allergy to one or more allergens is IgE driven food allergy.
A4. The anti-IgE antibody or antigen binding fragment thereof according to any one of embodiments A1-A3, wherein the one or more allergens is an allergen that induces food allergy or food intolerance in a subject.
A5. The anti-IgE antibody or antigen binding fragment thereof according to embodiment A4, wherein the one or more allergens that induces the food allergy or the food intolerance is present in food selected from the group consisting of: milk, peanut, a tree nuts, a cauliforate, a gluten containing grain crop, cheese, egg, shellfish, fish; and fruits.
A6. The anti-IgE antibody or antigen binding fragment thereof according to embodiment A3 wherein the food allergy is selected from the group consisting of a nut allergy, a fish allergy, a wheat allergy, a milk allergy, a peanut allergy, a tree nut allergy, a shellfish allergy, a soy allergy, a seed allergy, a sesame seed allergy, and an egg allergy.
A7. The anti-IgE antibody or antigen binding fragment thereof according to embodiment A6, wherein the food allergy is a peanut allergy.
A8. The anti-IgE antibody or antigen binding fragment thereof according to embodiment A6, wherein the food allergy is a milk allergy.
A9. The anti-IgE antibody or antigen binding fragment thereof according to embodiment A6, wherein the food allergy is an egg allergy.
A10. The anti-IgE antibody or antigen binding fragment thereof according to any one of embodiments A1-A9, for treating or preventing food allergy, e.g. severe food allergy, in an allergen-agnostic therapeutic approach.
A11. The anti-IgE antibody or antigen binding fragment thereof according to any one of embodiments A1-A10, for treating a food allergy, either by inducing non-specific tolerance against food allergens, or in a desensitization protocol, in combination with a food allergen.
A12. The anti-IgE antibody or antigen binding fragment thereof according to any one of embodiments A1-A10, wherein the food allergy is an IgE mediated food allergy.
A13. The anti-IgE antibody or antigen binding fragment thereof according to embodiment A12, wherein the IgE mediated food allergy is a severe food allergy.
A14. The anti-IgE antibody or antigen binding fragment thereof according to embodiment A12 or A13, wherein the IgE mediated food allergy is peanut allergens.
A15. The anti-IgE antibody or antigen binding fragment thereof according to any one of embodiments A1-A14, wherein an allergic reaction induced by the one or more allergens in a subject is reduced or abolished.
A16. The anti-IgE antibody or antigen binding fragment thereof according to any one of embodiments A1-A14 for use in preventing allergic reactions, e.g. allergic reactions including anaphylaxis, following accidental exposure to one or more allergens in a subject.
A17. The anti-IgE antibody or antigen binding fragment thereof according to any one of embodiments A1-A15 for use in the treatment of acute allergy and anaphylactic shock in a subject, following accidental exposure to one or more allergens.
A18. The anti-IgE antibody or antigen binding fragment thereof according to any one of embodiments A1-A14 for use in the management of an anaphylactic reaction from exposure to one or more food allergens in a subject.
A19. The anti-IgE antibody or antigen binding fragment thereof, e.g. ligelizumab, according to any one of embodiments A1-A14 for use in the treatment of severe allergic reactions to foods.
A20. The anti-IgE antibody or antigen binding fragment thereof, e.g. ligelizumab, according to any one of embodiments A1-A14 for use in the prevention of severe allergic reactions to foods, e.g. prevention to anaphylaxis reactions from accidental exposure to food allergens.
A21. The anti-IgE antibody or antigen binding fragment thereof according to any one of embodiments A1-A20 is to be administered repeatedly, e.g. during a cyclic period which lasts from about 1 month to about one year, which cyclic period may be repeated every two or three years, whereby the subject becomes tolerant to one or more food allergens.
A22. The anti-IgE antibody or antigen binding fragment thereof according to any one of embodiments A1-A21 for use in reducing the risk or severity of an allergic response or crisis.
A23. The anti-IgE antibody or antigen binding fragment thereof according to any one of embodiments A1-A22, wherein food allergy is peanuts allergy, milk allergy or egg allergy.
A24. The anti-IgE antibody or antigen binding fragment thereof according to any one of embodiments A1-A23, wherein the anti-IgE antibody or antigen binding fragment thereof is administered to the subject less than 4 hours after manifestation of one or more allergic symptoms in the subject.
A25. The anti-IgE antibody or antigen binding fragment thereof according to embodiment A24, wherein the one or more allergic symptoms are selected from the group consisting of urticaria, angioedema, rhinitis, asthma, vomiting, respiratory compromise, swollen lips, swollen tongue, and reduced blood pressure.
A26. An anti-IgE antibody or antigen binding fragment thereof for use in treating, preventing or reducing allergic reaction in a subject in need thereof, wherein the allergic reaction is anaphylaxis.
A27. An anti-IgE antibody or antigen binding fragment thereof according to embodiment 26, wherein the allergic reaction is triggered by consumption of or exposure to a food item.
A28. An anti-IgE antibody or antigen binding fragment thereof according to embodiment 26, wherein the allergic reaction is triggered by exposure to a non-food allergen.
A29. An anti-IgE antibody or antigen binding fragment thereof for use in treating, preventing or reducing the severity of allergic reactions in a subject in need thereof.
A30. An anti-IgE antibody or antigen binding fragment thereof for use in the prevention of allergic reactions, including anaphylaxis, following accidental exposure to food allergens in a subject with a confirmed diagnosis of IgE driven food allergy to one or more allergens.
A31. An anti-IgE antibody or antigen binding fragment thereof for use in the prevention of signs and symptoms of anaphylaxis during an oral food challenge in food-allergic subject.
A32. The anti-IgE antibody or antigen binding fragment thereof according to any of the above embodiments, wherein the anti-IgE antibody is ligelizumab.
A33. The anti-IgE antibody or antigen binding fragment thereof according to embodiment A32, wherein ligelizumab is administered at a dose of about 24 mg to about 600 mg, e.g. at a maximum dose of 600 mg.
A34. The anti-IgE antibody or antigen binding fragment thereof according to embodiment A32 or embodiment A33, wherein ligelizumab is administered at a dose of about 120 mg.
A35. The anti-IgE antibody or antigen binding fragment thereof according to embodiment A32 or embodiment A33, wherein ligelizumab is administered at a dose of about 240 mg.
A36. The anti-IgE antibody or antigen binding fragment thereof according to embodiment A33, wherein ligelizumab is administered at a dose of about 300 mg.
A37. The anti-IgE antibody or antigen binding fragment thereof according to embodiment A33, wherein ligelizumab is administered at a dose of about 360 mg.
A38. The anti-IgE antibody or antigen binding fragment thereof according to embodiment A33, wherein ligelizumab is administered at a dose of about 480 mg.
A39. The anti-IgE antibody or antigen binding fragment thereof according to embodiments 32 to A38, wherein ligelizumab is administered every two to four weeks.
A40. The anti-IgE antibody or antigen binding fragment thereof according to any one of embodiments A32 to A39, wherein ligelizumab is administered during up to 16 weeks, e.g. 12 to 16 weeks.
A41. The anti-IgE antibody or antigen binding fragment thereof according to any one of embodiments A32 to A40, wherein ligelizumab is administered as monotherapy to said subject.
A42. The anti-IgE antibody or antigen binding fragment thereof according to any of the above embodiments, wherein the anti-IgE antibody is co-administered with OIT.
A43. The anti-IgE antibody or antigen binding fragment thereof according to any of the above embodiments, wherein the anti-IgE antibody is administered as adjuvant to OIT.
A44. An anti-IgE antibody or antigen binding fragment thereof, e.g. ligelizumab, for use in treating, preventing or reducing food allergy, such as severe food allergy, to one or more allergens in a subject in need thereof, wherein ligelizumab is administered as monotherapy to said subject.
A45. An anti-IgE antibody or antigen binding fragment thereof, e.g. ligelizumab, for use in preventing allergic reactions, such as anaphylaxis, one or more allergens in a subject in need thereof, wherein ligelizumab is administered as monotherapy to said subject.
A46. An anti-IgE antibody or antigen binding fragment thereof, e.g. ligelizumab, for use in treating or preventing food allergy, such as severe food allergy, to one or more allergens in a subject in need thereof, wherein ligelizumab is administered to said subject in combination with OIT treatment or as adjuvant to OIT.
A47. An anti-IgE antibody or antigen binding fragment thereof, e.g. ligelizumab, for use in preventing allergic reactions, such as anaphylaxis, one or more allergens in a subject in need thereof, wherein ligelizumab is administered as to said subject in combination with OIT treatment or as adjuvant to OIT.
A48. The anti-IgE antibody or antigen binding fragment thereof according to any of the above embodiments, wherein the anti-IgE antibody or antigen binding fragment thereof is used in a method for preventing or treating one or more symptoms associated with food allergy.
A49. A pharmaceutical composition comprising the anti-IgE antibody or antigen binding fragment thereof according to any of the above embodiments.

### Definitions

As used herein, IgE refers to Immunoglobulin E.

The term “comprising” encompasses “including” as well as “consisting,” e.g., a composition “comprising” X may consist exclusively of X or may include something additional, e.g., X+Y.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “antibody” as referred to herein includes naturally-occurring and whole antibodies. A naturally-occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed hypervariable regions or complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody, as used herein, refers to fragments of an antibody that retain the ability to specifically bind to IgE. It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated CDR. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies are also intended to be encompassed within the term “antibody”. Single chain antibodies and antigen-binding portions are obtained using conventional techniques known to those of skill in the art.

Exemplary antibodies include ligelizumab antibody (U.S. Pat. No. 7,531,169), the disclosure of which is incorporated by reference herein in their entirety.

The term “antigen-binding fragment” of an antibody, as used herein, refers to fragments of an antibody that retain the ability to specifically bind to an antigen (e.g., IgE). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated CDR. Exemplary antigen-binding fragments include the CDRs of ligelizumab having a variable light chain region comprising CDRL1, CDRL2, and CDRL3 and a variable heavy chain region comprising CDRH1, CDRH2, and CDRH3, wherein CDRL1 consists of SEQ ID NO:3, CDRL2 consists of SEQ ID NO:4, CDRL3 consists of SEQ ID NO:5, CDRH1 consists of SEQ ID NO:6, CDRH2 consists of SEQ ID NO:7, and CDRH3 consists of SEQ ID NO:8, wherein the antibody binds specifically to IgE.

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies are also intended to be encompassed within the term “antibody”. Single chain antibodies and antigen-binding portions are obtained using conventional techniques known to those of skill in the art.

An “isolated antibody”, as used herein, refers to an antibody that is substantially free of other antibodies having different antigenic specificities (e.g., an isolated antibody that specifically binds IgE is substantially free of antibodies that specifically bind antigens other than IgE). The term “monoclonal antibody” or “monoclonal antibody composition” as used herein refer to a preparation of antibody molecules of single molecular composition. The term “human antibody”, as used herein, is intended to include antibodies having variable regions in which both the framework and CDR regions are derived from sequences of human origin. A “human antibody” need not be produced by a human, human tissue or human cell. The human antibodies of the disclosure may include amino acid residues not encoded by human sequences (e.g., mutations introduced by random or site-specific mutagenesis in vitro, by N-nucleotide addition at junctions in vivo during recombination of antibody genes, or by somatic mutation in vivo). In some embodiments of the disclosed processes and compositions, the anti-IgE antibody is a human antibody, an isolated antibody, and/or a monoclonal antibody.

As used herein, “anti-human IgE antibody” means an antibody that binds to human IgE in such a manner so as to inhibit or substantially reduce the binding of such IgE to the high affinity receptor, FcεRI.

The term “K_{D}” is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, or using a biosensor system such as a Biacore® system. In some embodiments, the anti-IgE antibody or antigen-binding fragment thereof according to the invention, e.g., omalizumab, binds human IgE with a K_{D} of about 0.02 to 7.7 nM, e.g. 100-250 pM.

The term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. Standard assays to evaluate the binding affinity of the antibodies toward IgE of various species are known in the art, including for example, ELISAs, western blots and RIAs. The binding kinetics (e.g., binding affinity) of the antibodies also can be assessed by standard assays known in the art, such as by Biacore analysis.

An antibody that “inhibits” one or more IgE functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (or when a control antibody of irrelevant specificity is present). An antibody that inhibits IgE activity affects a statistically significant decrease, e.g., by at least about 10% of the measured parameter, by at least 50%, 80% or 90%, and in certain embodiments of the disclosed methods and compositions, the IgE antibody used may inhibit greater than 95%, 98% or 99% of IgE functional activity.

The term “derivative”, unless otherwise indicated, is used to define amino acid sequence variants, and covalent modifications (e.g. pegylation, deamidation, hydroxylation, phosphorylation, methylation, etc.) of an anti-IgE antibody or antigen-binding fragment thereof, e.g., ligelizumab, according to the present disclosure, e.g., of a specified sequence (e.g., a variable domain). A “functional derivative” includes a molecule having a qualitative biological activity in common with the disclosed anti-IgE antibodies. A functional derivative includes fragments and peptide analogs of an anti-IgE antibody as disclosed herein. Fragments comprise regions within the sequence of a polypeptide according to the present disclosure, e.g., of a specified sequence.

The phrase “substantially identical” means that the relevant amino acid or nucleotide sequence (e.g., V_{H} or V_{L} domain) will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region (e.g., V_{H} or V_{L} domain). In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity of a derivative anti-IgE antibody (e.g., a derivative of ligelizumab, e.g., an ligelizumab biosimilar antibody) can be about 90% or greater, e.g., 90%, 91%, 92%, 93%, 94%, 95%, 96%, 97%, 98%, 99% or higher relative to the disclosed sequences.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are well known. The percent identity can be determined by standard alignment algorithms, for example, the Basic Local Alignment Search Tool (BLAST) described by Altshul et al. ((1990) J. Mol. Biol., 215: 403 410); the algorithm of Needleman et al. ((1970) J. Mol. Biol., 48: 444 453); or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

“Amino acid(s)” refer to all naturally occurring L-α-amino acids, e.g., and include D-amino acids. The phrase “amino acid sequence variant” refers to molecules with some differences in their amino acid sequences as compared to the sequences according to the present disclosure. Amino acid sequence variants of an antibody according to the present disclosure, e.g., of a specified sequence, still have the ability to bind the IgE. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The term “pharmaceutically acceptable” means a nontoxic material that does not interfere with the effectiveness of the biological activity of the active ingredient(s).

The term “administering” in relation to a compound, e.g., an anti-IgE antibody, is used to refer to delivery of that compound to a subject by any route.

As used herein, a “therapeutically effective amount” refers to an amount of anti-IgE antibody (e.g., ligelizumab or an antigen-binding fragment thereof) that is effective, upon single or multiple dose administration to a subject (such as a human) for treating, preventing, preventing the onset of, curing (if applicable), delaying, reducing the severity of, ameliorating at least one symptom of a disorder or recurring disorder, or prolonging the survival of the subject beyond that expected in the absence of such treatment. When applied to an individual active ingredient (e.g., an anti-IgE antibody, e.g., ligelizumab) administered alone, the term refers to that ingredient alone. When applied to a combination, the term refers to combined amounts of the active ingredients that result in the therapeutic effect, whether administered in combination, serially or simultaneously.

The term “treatment” or “treat” is herein defined as the application or administration of an anti-IgE antibody according to the disclosure, for example, ligelizumab, or a pharmaceutical composition comprising said anti-IgE antibody, to a subject or to an isolated tissue or cell line from a subject, where the subject has a particular disease, a symptom associated with the disease, or a predisposition towards development of the disease, where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a subject suspected to have the disease as well as subjects who are ill or who have been diagnosed as suffering from the disease or medical condition, and includes suppression of clinical relapse.

Furthermore, the term “treatment” or “treat” is herein defined as the application or administration of an IgE antibody according to the disclosure, for example ligelizumab, or a pharmaceutical composition comprising said anti-IgE antibody, to a subject or to an isolated tissue or cell line from a subject, where the subject has a particular disease (e.g., anaphylaxis), a symptom associated with the disease (e.g., anaphylaxis), or a predisposition towards development of the disease (e.g., anaphylaxis) (if applicable), where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a subject suspected to have the disease as well as subjects who are ill or who have been diagnosed as suffering from the disease or medical condition, and includes suppression of clinical relapse.

“Effective treatment” is herein defined as the application or administration of an IgE antibody according to the disclosure, for example ligelizumab, or a pharmaceutical composition comprising said anti-IgE antibody, to a subject where the subject has a achieved a total IgE <20 IU/mL upon administration of ligelizumab.

Also, “Effective treatment” is herein defined as the application or administration of an IgE antibody according to the disclosure, for example ligelizumab, or a pharmaceutical composition comprising said anti-IgE antibody, to a subject where the subject can tolerate a single dose of 3000 mg (5044 mg cumulative tolerated dose) of peanut protein without dose-limiting symptoms during the DBPCFC, or can tolerate a single dose of z 1000 mg (2044 mg cumulative tolerated dose) of peanut protein without dose-limiting symptoms during the DBPCFC, or can tolerate a single dose of 3000 mg (5044 mg cumulative tolerated dose) of peanut protein without dose-limiting symptoms during the DBPCFC, upon administration of ligelizumab.

As used herein, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By selecting, selectively treating and selectively administering, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology. In some embodiments, the patient was selected for treatment based on having food allergy.

As used herein, the phrase “population of subjects” is used to mean a group of subjects. In some embodiments of the disclosed methods, the IgE antagonist (e.g., anti-IgE antibody, such as ligelizumab) is used to treat a population of subjects with IgE mediated allergic reaction to one or more allergens.

As herein defined, the term “allergic reaction” is defined as a hypersensitivity reaction initiated by proven or strongly suspected immunologic mechanisms.

As herein defined, the term “food allergy”, is used when a causal relationship (ideally, with a specific immunological mechanism) has been defined.

There are three broad groups of immune reactions: IgE-mediated, non-IgE-mediated and mixed. The IgE-mediated reactions are usually divided into immediate-onset reactions (arising up to 2 hours from ingestion of, or exposure to, or contact with the offending triggers, e.g. food(s)) and immediate plus late-phase (in which the immediate onset symptoms are followed by prolonged or ongoing symptoms). Non-IgE-mediated reactions, which are poorly defined both clinically and scientifically, are believed to be generally T-cell-mediated. They are typically delayed in onset, and occur 4 to 28 hours after ingestion of, or exposure to, or contact with the offending triggers, e.g. food(s). Mixed IgE and non-IgE mediated reaction are conditions associated with, e.g. food allergy, involving both IgE- and non-IgE-mediated mechanisms.

As herein defined, the term “severe food allergy” refers to a severe reaction to one or more food allergens including any one of the following symptoms: anaphylaxis, low blood pressure, trouble breathing, or wheezing; or combinations of multiple organ symptoms, including vomiting, angioedema, and/or coughing in combination. In some examples, “severe food allergy” refers to food allergy wherein a subject experiences at least one stringent symptom affecting at least two of the following organ systems: skin or oral mucosa, gastrointestinal tract, cardiovascular and respiratory tract.

As herein defined, the term “food allergens” refers to allergens present in certain foods, such as e.g., milk, peanut, tree nuts, cauliforate, grain crop, cheese, egg, shellfish, fish and fruits.

As herein defined, the term “IgE mediated (or driven) food allergy to one or more allergens” refers to e.g. food allergy. In some embodiments, food allergy refers to food allergy to one or more allergens, wherein the one or more allergens that induces food allergy or food intolerance is present in food selected from the group consisting of: milk, peanut, a tree nuts, a cauliforate, a gluten containing grain crop, cheese, egg, shellfish, fish; and fruits. In some embodiments, food allergy refers to, e.g. peanuts, milk or egg allergy. In some embodiments, food allergy also refers to an indication for the prevention of allergic reactions, including anaphylaxis, following accidental exposure to food allergens in a subject, e.g. an adult and pediatric patients 6 years of age and older, with a confirmed diagnosis of IgE mediated food allergy to one or more allergens.

As herein defined, the term “symptoms associated with food allergy” refers to symptoms related to any of the following organ systems: gastrointestinal tract, cutaneous system, respiratory system or cardiovascular system. Such symptoms can be, but are not limited to, e.g., vomiting, pain, or diarrhea; urticaria, angioedema, or pruritus; acute rhinoconjunctivitis, wheezing, coughing or stridor; collapse as a result of hypotension.

As herein defined, the term “allergen-agnostic” treatment or therapeutic approach refers to allergy treatment or allergy therapeutic approach that is not dependent on the allergen.

A double blind, placebo-controlled food challenge (DBPCFC) is the preferred test to diagnose food allergy.

Oral immunotherapy (OIT) is a medical treatment in which an individual who is allergic to a specific food consumes an increasing amount of the allergen with the goal of reducing the risk of allergic reactions to the food. Oral immunotherapy is in general composed of multiple allergenic proteins (allergen). An example of an OIT is the oral peanut (Arachis hypogaea) allergen powder, known under the product name Palforzia. Multi-allergen OIT may contain more than one allergenic protein (food protein flours), e.g. more than one of the following food protein flours: peanut, milk, egg, wheat, cashew, hazelnut, and walnut (all food protein flours).

According to World Allergy Organization Anaphylaxis Guidance 2020, anaphylaxis is defined as a severe, life-threatening systemic hypersensitivity reaction characterized by being rapid in onset with potentially life-threatening airway, breathing, or circulatory problems and is usually, although not always, associated with skin and mucosal changes. A large variety of molecules can induce anaphylaxis. These are most frequently proteins, which induce anaphylaxis in an IgE-dependent manner or molecules, which directly activate mast cells. An IgE-mediated anaphylaxis is triggered by the interaction of an allergen (usually a protein) interacting with the allergen-specific IgE/high-affinity receptor (FcεRI) complex expressed on effector cells, predominantly mast cells and basophils. Anaphylaxis is classified as idiopathic when no trigger can be identified.

Non-responders to therapy using an anti-IgE antibody or antigen-binding fragment thereof, are defined as subjects who failed to achieve a 90% improvement of their baseline or had an exacerbation of their symptoms. Responders to therapy using an anti-IgE antibody or antigen-binding fragment thereof are defined as those subjects who achieved 90% improvement of baseline.

### Anti-IgE Antibodies

In some embodiments of the disclosed uses, methods, and kits, the anti-IgE antibody or antigen-binding fragment thereof is a monoclonal antibody. In some embodiments, the anti-IgE antibody or antigen-binding fragment thereof is a human or humanized antibody. In some embodiments, the anti-IgE antibody or antigen-binding fragment thereof is a humanized antibody. In some embodiments, the anti-IgE antibody or antigen-binding fragment thereof is a human antibody of the IgG, subtype. In other embodiments, the anti-IgE antibody or antigen-binding fragment thereof is ligelizumab.

Exemplary anti-IgE antibodies include, but are not limited to, omalizumab, quilizumab, ligelizumab and etrolizumab.

Alternatively, an anti-IgE antibody or antigen-binding fragment thereof used in the disclosed methods may be an amino acid sequence variant of the reference anti-IgE antibodies set forth herein.

The disclosure also includes anti-IgE antibodies or antigen-binding fragments thereof (e.g., ligelizumab) in which one or more of the amino acid residues of the V_{H} or V_{L} domain of ligelizumab, typically only a few (e.g. 1-10), are changed; for instance by mutation, e.g., site directed mutagenesis of the corresponding DNA sequences.

In one embodiment, the anti-IgE antibody or antigen-binding fragment thereof comprises an immunoglobulin VH domain comprising the amino acid sequence set forth as SEQ ID NO:2 and an immunoglobulin VL domain comprising the amino acid sequence set forth as SEQ ID NO:1SEQ ID NO:1.

In one embodiment, the anti-IgE antibody or antigen-binding fragment thereof comprises a variable light chain region comprising CDRL1, CDRL2, and CDRL3 and a variable heavy chain region comprising CDRH1, CDRH2, and CDRH3, wherein CDRL1 consists of SEQ ID NO:3, CDRL2 consists of SEQ ID NO:4, CDRL3 consists of SEQ ID NO:5, CDRH1 consists of SEQ ID NO:6, CDRH2 consists of SEQ ID NO:7, and CDRH3 consists of SEQ ID NO:8, wherein the antibody binds specifically to IgE.

Alternatively, an anti-IgE antibody or antigen-binding fragment thereof as used in the disclosed methods may comprise a derivative of the anti-IgE antibodies set forth herein by sequence (e.g., pegylated variants, glycosylation variants, affinity-maturation variants, etc.). Alternatively, the V_{H} or V_{L} domain of an anti-IgE antibody or antigen-binding fragment thereof used in the disclosed methods may have V_{H} or V_{L} domains that are substantially identical to the V_{H} or V_{L} domains set forth herein (e.g., those set forth in SEQ ID NO:2 and 61). A human anti-IgE antibody disclosed herein may comprise a heavy chain that is substantially identical to that set forth as SEQ ID NO:2 and/or a light chain that is substantially identical to that set forth as SEQ ID NO:1. A human anti-IgE antibody disclosed herein may comprise a heavy chain that comprises SEQ ID NO:2 and a light chain that comprises SEQ ID NO:1.

The preferred anti-IgE antibodies or antigen-binding fragments thereof used in the disclosed methods are human antibodies, especially ligelizumab as described in Table 2 of Examples 10 of U.S. Pat. No. 7,531,169, which is incorporated by reference herein in its entirety.

### Methods of Treatment and Uses of Anti-IgE Antibodies

The symptoms and severity of an allergy, e.g. food allergy, may depend on factors such as type of immune response(s) involved, the duration and magnitude of the immune response(s), amount of allergen, and the site of contact/exposure to the allergen. Examples of allergy symptoms include, without limitation, skin rash, skin redness, hives, skin bumps/patches/welts, itchy/watery eyes, headache, sneezing, wheezing, shortness of breath, chest tightness, cough, runny nose, sore throat, swelling, nausea, vomiting, diarrhea, and anaphylaxis.

A subject may contact or be exposed to an allergen that induces an allergic reaction by any route known in the art, for example, through ingestion, inhalation, injection, or direct contact. The symptoms associated with the allergic reaction may be localized to the site of contact or exposure to the allergen, for example a region of the skin, respiratory tract, or gastrointestinal tract, a distal site, or may become systemic, such as in the case of anaphylaxis.

In general, an allergic reaction may occur immediately after contact or exposure to an allergen or within about a half-hour or longer after contact or exposure.

Although there is a broad variety of known food allergens, over 90 percent of adverse food reactions are caused by food components in the following foods: milk, eggs, peanuts, tree nuts, wheat, soy, fish, and shellfish. Other examples of food allergies are allergies caused by food components in legumes (soy, peas, beans), corn, maize, fruits, vegetables, spices, synthetic and natural colors, chicken and chemical additives.

One of the most common food allergies is peanut allergy. Peanuts belong to the family of legumes (Fabaceae). Proteins in tree nuts, including pecans, almonds, cashews, pistachios, pine nuts, and walnuts, are another widespread allergen. Subjects suffering from tree nut allergy may be sensitive to one, or many, tree nuts. Furthermore, seeds, including sesame seeds and poppy seeds, may contain oils comprising a protein that can act as an allergen. In particular embodiments, the food allergy may be thus selected from the group consisting of peanut allergy, milk allergy, nut allergy, corn allergy, fruit allergy, garlic allergy, oats allergy, shellfish allergy, soy allergy, wheat allergy (in particular gluten allergy), egg allergy, sesame allergy, olive oil allergy, cheese allergy, crustaceans allergy, fish allergy. Milk allergy may be further distinguished by the animal (cow, goat etc.) from which the milk originates.

Food allergies include immediate (IgE mediated) food allergies, and (IgE/non-IgE and non-IgE mediated) delayed food allergies. Immediate (IgE mediated food) allergy can affect many systems of the body (skin, gut, airway and circulation) and symptoms develop rapidly (within one hour) of eating the food. In contrast, delayed forms of food allergy mainly affect the bowel and the skin and symptoms develop hours after eating the food. The immune mechanisms causing delayed food allergy are less well understood than IgE-mediated food allergy. The most common causative foods for delayed food allergies are cow's milk and soy. Unlike IgE mediated food allergy, delayed food allergies are very rarely life threatening. The present invention encompasses treating or preventing food allergy or food intolerance; e.g. an immediate food allergy.

Examples of allergies that can be treated according to the anti-IgE antibodies and methods provided herein, include without limitation, food allergy, e.g. IgE mediated food allergy. In some embodiments, the allergy is a food allergy selected from the group consisting of peanut allergy, milk allergy, nut allergy, corn allergy, fruit allergy, garlic allergy, oats allergy, shellfish allergy, soy allergy, wheat allergy (in particular gluten allergy), egg allergy, sesame allergy, olive oil allergy, cheese allergy, crustaceans allergy, fish allergy.

Several common allergenic foods (milk, egg, and wheat) commonly cause IgE-mediated and non-IgE-mediated or mixed IgE-mediated and non-IgE-mediated reactions, but others (peanut, sesame, and shellfish) nearly always cause only IgE-mediated reactions.

Some specific food-induced allergic conditions on the basis of pathophysiology are described here below:
(i) IgE-mediated (acute onset):
   Acute urticaria/angioedema (Food commonly causes acute (20%) but rarely chronic urticaria), Most common causal foods: primarily “major allergens”;
   Contact urticaria (Direct skin contact results in lesions. Rarely this is due to direct histamine release (nonimmunologic)). Most common causal foods:multiple:
   Anaphylaxis (Rapidly progressive, multiple organ system reaction can include cardiovascular collapse). Most common causal foods: Any but more commonly peanut, tree nuts, shellfish, fish, milk, and egg;
   Food-associated, exercise-induced anaphylaxis (Food triggers anaphylaxis only if ingestion is followed temporally by exercise). Most common causal foods: Wheat, shellfish, and celery most often described;
   Oral allergy syndrome (pollen-associated food allergy syndrome) (Pruritus and mild edema are confined to oral cavity and uncommonly progress beyond the mouth (7%) and rarely to anaphylaxis (1% to 2%)) Might increase after pollen season. Most common causal foods: Raw fruit/vegetables; cooked forms tolerated; examples of relationships: birch (apple, peach, pear, carrot), ragweed (melons);
   Immediate gastrointestinal hypersensitivity (Immediate vomiting, pain) Most common causal foods: Major allergen;
(ii) Combined IgE- and cell-mediated (delayed onset/chronic)
   Atopic dermatitis (Associated with food allergy in; 35% of children with moderate-to-severe rash) Most common causal foods: Major allergens, particularly egg, milk
   Eosinophilic esophagitis (Symptoms might include feeding disorders, reflux symptoms, vomiting, dysphagia, and food impaction). Most common causal foods: Multiple.
   Eosinophilic gastroenteritis (Vary on site(s)/degree of eosinophilic inflammation; might include ascites, weight loss, edema, obstruction) Most common causal foods: Multiple.

As used herein, the expressions “oral immunotherapy”, “OIT”, or “OIT regimen,” and the like, refer to the repeated administration of an allergen to a subject over time as means for treating or preventing allergies and allergic reactions, or to reduce or eliminate allergic responses. In a typical OIT regimen, small amounts of allergen are initially administered to an allergic subject, followed by administration of increased amounts of allergen. In certain instances, the OIT regimen comprises at least two consecutive phases: (1) an up-dosing phase, and (2) a maintenance phase. In the up-dosing phase, increasing doses of allergen are administered until an effective and safe dose is achieved. The dose that is established at the end of the up-dosing phase is then administered to the subject throughout the course of the maintenance phase. The duration of the up-dosing phase can be several weeks or several months. In certain embodiments, however, the up-dosing phase is of substantially shorter duration (e.g., less than one week, less than 6 days, less than 5 days, less than 4 days, less than 3 days, or less than 2 days). The maintenance phase of an OIT regimen can last several weeks, several months, several years, or indefinitely.

According to the methods of the present invention, ligelizumab can be administered to the subject throughout the entire course of the OIT regimen, or for only a portion of the OIT regimen. For example, the methods of the present invention include administration of a therapeutically effective amount of a pharmaceutical composition comprising ligelizumab to a subject at a frequency of about once a week, once every two weeks, once every three weeks, once a month, once every two months, once every four months, once every six months, or less frequently, prior to or during the up-dosing phase. In certain embodiments, the pharmaceutical composition comprising ligelizumab is administered to the subject at a frequency of about once a week, once every two weeks, once every three weeks, once a month, once every two months, once every four months, once every six months, or less frequently, during or after the maintenance phase.

According to the present invention, the efficacy and/or safety of an OIT regimen is “enhanced” if one or more of the following outcomes or phenomena are observed or achieved in a subject: (1) the duration of the up-dosing phase is decreased without compromising efficacy or safety; (2) the duration of the maintenance phase is decreased without compromising efficacy or safety; (3) the number of doses of allergen administered during the up-dosing or maintenance phase is reduced without compromising efficacy or safety; (4) the frequency of allergen administration during the up-dosing or maintenance phase is reduced without compromising efficacy or safety; (5) the dose of allergen administered during the up-dosing or maintenance phase is increased without compromising efficacy or safety; (6) the frequency of allergic responses or adverse side-effects triggered by the OIT regimen is reduced or eliminated; (7) the use of or need for conventional allergy medications (e.g., steroids, antihistamines, decongestants, anti-IgE agents, etc.) is reduced or eliminated during the up-dosing and/or maintenance phases; (8) the level of allergen-induced IgE expression is reduced; and/or (9) the frequency of anaphylactic reactions is reduced or eliminated. The efficacy of an OIT regimen is also deemed to be “enhanced,” according to the present invention, if a subject experience fewer and/or less severe allergic reactions following OIT therapy in combination with anti-IgE antibody than with OIT therapy alone.

### Anti-IgE Antibody

Antibodies against IgE (“anti-IgE antibodies”) are known from e.g. in U.S. Pat. No. 7,531,169 as Mab 2 (CL-2C) and defined by SEQ ID NOs: 61 and 62, which is incorporated by reference herein in its entirety. This antibody is referred to hereafter as QGE031, or under its international non-proprietary name ligelizumab.

The disclosed anti-IgE antibody (e.g., ligelizumab) or antigen-binding fragment thereof, may be used in vitro, ex vivo, or incorporated into pharmaceutical compositions and administered in vivo to treat subjects (e.g., human subjects) affected by one or more IgE driven food allergy to one or more allergens, e.g. food allergy, peanuts allergy.

The anti-IgE antibody (e.g., ligelizumab) or antigen-binding fragment thereof may be used as a pharmaceutical composition when combined with a pharmaceutically acceptable carrier. Such a composition may contain, in addition to the anti-IgE antibody or antigen-binding fragment thereof, carriers, various diluents, fillers, salts, buffers, stabilizers, solubilizers, and other materials well known in the art. The characteristics of the carrier will depend on the route of administration.

Pharmaceutical compositions for use in the disclosed methods may be manufactured in conventional manner. In one embodiment, the pharmaceutical composition is provided in lyophilized form. For immediate administration it is dissolved in a suitable aqueous carrier, for example sterile water for injection or sterile buffered physiological saline. Other formulations comprise liquid or lyophilized formulation.

Antibodies, e.g., antibodies to IgE, or antigen-binding fragment thereof, are typically formulated either in aqueous form ready for parenteral administration or as lyophilisates for reconstitution with a suitable diluent prior to administration. In some embodiments of the disclosed methods and uses, the anti-IgE antibody or antigen-binding fragment thereof, e.g., ligelizumab, is formulated as a lyophilisate. Suitable lyophilisate formulations can be reconstituted in a small liquid volume (e.g., 2 ml or less, e.g., 1 ml) to allow subcutaneous administration and can provide solutions with low levels of antibody aggregation. Techniques for purification of antibodies to a pharmaceutical grade are well known in the art.

Disclosed methods of, and anti-IgE antibodies for use in, preventing, treating or modifying the course of a IgE driven food allergy to one or more allergens, e.g. food allergy, peanuts allergy in a subject in need thereof, comprising herein are administering the subject a therapeutically effective amount of an anti-IgE antibody or antigen-binding fragment thereof.

For example, IgE driven food allergy to one or more allergens is food allergy, e.g. peanuts allergy.

In yet another example, the IgE driven food allergy to one or more allergens disease or disorder refers to peanuts allergy, milk allergy or egg allergy.

Disclosed herein are methods of, and anti-IgE antibodies (e.g. ligelizumab) for use in, preventing, treating or modifying the course of a disease or disorder involving allergic reactions to one or more allergens driven by IgE, in a subject in need thereof, comprising administering the subject a therapeutically effective amount of anti-IgE antibody or antigen-binding fragment thereof.

In another embodiment, the subject is affected by food allergies, e.g. peanuts allergy.

Ligelizumab demonstrated dose- and time-dependent suppression of free IgE, basophil FcεRI, basophil surface IgE, and skin prick test responses to allergen, superior in extent and duration to those observed with omalizumab. Superior affinity and pharmacodynamic (PD) outcomes of ligelizumab compared to omalizumab may translate into superior posology and superior clinical efficacy in subjects. Ligelizumab binds to IgE with 150fold higher affinity than omalizumab (A. Eggel, Molecular, Structural And Mechanistic Insight Into Ligelizumab Mediated Suppression Of IgE Dependent Allergic Responses, EAACI 2019).

Furthermore, disclosed herein are anti-IgE antibodies or antigen-binding fragments thereof, e.g. ligelizumab, for use in, preventing, treating or modifying the course of a disease or disorder which is IgE driven food allergy to one or more allergens, e.g. food allergy, peanuts allergy, in a subject in need thereof, comprising administering the subject a therapeutically effective amount of anti-IgE antibody or antigen-binding fragment thereof, wherein the subject is not affected by allergy, asthma, urticarial or rhinitis.

The appropriate dosage will vary depending upon, for example, the particular anti-IgE antibody to be employed, the host, the mode of administration and the nature and severity of the condition being treated, and on the nature of prior treatments that the subject has undergone. It may also depend on the level of IgE in the subject's blood before initiating the treatment with the anti-IgE antibody.

Ultimately, the attending health care provider will decide the amount of the anti-IgE antibody with which to treat each individual subject. In some embodiments, the attending health care provider may administer low doses of the anti-IgE antibody and observe the subject's response, in particular the blood level of IgE.

In one embodiment of the present disclosure, ligelizumab is administered subcutaneously at a dose of about 24 mg to about 600 mg, at a dose of about 24 mg to about 240 mg, e.g. at a dose of about 24 mg, of about 72 mg, of about 120 mg, of about 240 mg, of about 300 mg, of about 360 mg, or of about 480 mg, e.g. at a maximum dose of about 600 mg.

In some embodiments, the level of IgE in the subject's blood is measured before initiating the administration of the anti-IgE antibody, e.g., ligelizumab, and the dose of the antibody is adjusted based on the weight of the subject and/or his serum total IgE level.

The duration of therapy using a pharmaceutical composition of the present disclosure will vary, depending on the severity of the disease or disorder to be treated and the condition and personal response of each individual subject. In some embodiments, the subject is administered the anti-IgE antibody (e.g., ligelizumab) for long-term, e.g. at least 12 weeks, e.g. up to 16 weeks, e.g. to 12 to 16 weeks.

In some embodiments, the anti-IgE antibody (e.g., ligelizumab) is administered to the subject every two weeks, e.g. every two or four weeks, e.g. monthly.

The anti-IgE antibody or antigen-binding fragment thereof according to the present disclosure, e.g., ligelizumab, is conveniently administered parenterally, e.g., intravenously, intramuscularly, or subcutaneously, e.g. subcutaneously.

The anti-IgE antibody or antigen-binding fragment thereof, e.g., ligelizumab, may be administered to the subject subcutaneously (SC), e.g. at about 24 mg to about 600 mg (e.g. about 24 mg, about 72 mg, about 120 mg, about 240 mg, or about 600 mg), e.g. at about 120 mg.

The anti-IgE antibody or antigen-binding fragment thereof, e.g., ligelizumab, may be administered to the subject subcutaneously (SC), e.g. at about 300 mg to about 360 mg, e.g. at about 360 mg.

The anti-IgE antibody or antigen-binding fragment thereof, e.g., ligelizumab, may be administered to the subject subcutaneously (SC), e.g. at about 400 mg to about 600 mg, e.g. at about 480 mg.

In practicing some of the methods of treatment or uses of the present disclosure, a therapeutically effective amount of an anti-IgE antibody (e.g., ligelizumab) or antigen-binding fragment thereof, is administered to a subject, e.g., a mammal (e.g., a human). While it is understood that the disclosed methods provide for treatment of diseases or disorders involving IgE, using anti-IgE antibody (e.g., ligelizumab) or antigen-binding fragment thereof this does not preclude that, if the subject is to be ultimately treated with an anti-IgE antibody (e.g., ligelizumab) or antigen-binding fragment thereof, therapy is necessarily a monotherapy.

Indeed, if a subject is selected for treatment with an anti-IgE antibody or antigen-binding fragment thereof, then the anti-IgE antibody (e.g., ligelizumab) or antigen-binding fragment thereof, may be administered in accordance with the methods of the disclosure either alone or in combination with other agents and therapies for treating the subject affected by the disease or disorder involving IgE, e.g., in combination with at least one additional therapeutic agent, such as e.g., a corticosteroid or an immunosuppressor, e.g., a systemic corticosteroid or an immunosuppressor.

That can be the case for example, when the subject to be treated is allergic, or when the subject is also affected by another disease or disorder selected from asthma, urticaria, and rhinitis, e.g. selected from allergic asthma, CSU, and allergic rhinitis.

When coadministered with one or more additional food allergy agent(s), the anti-IgE antibody or antigen-binding fragment thereof may be administered either simultaneously with the other agent, or sequentially. If administered sequentially, the attending physician will decide on the appropriate sequence of administering the anti-IgE antibody or antigen-binding fragment thereof in combination with other agents and the appropriate dosages for co-delivery. Various therapies may be beneficially combined with the disclosed anti-IgE antibodies, such as ligelizumab, during treatment of the disease or disorder involving IgE, disclosed herein. Such therapies include for example corticosteroids (e.g., systemic corticosteroids) or immunosuppressors.

Disclosed herein are methods of, and anti-IgE antibodies (e.g., ligelizumab) or antigen-binding fragment thereof, for use in, modifying the course of a disease or disorder disclosed herein, e.g. IgE driven food allergy to one or more allergens is food allergy, e.g. peanuts allergy, in a subject in need thereof, comprising administering the subject a dose of about 24 mg to about 600 mg of an anti-IgE antibody (e.g., ligelizumab) or antigen-binding fragment thereof by subcutaneous injection.

Disclosed herein are methods of, and anti-IgE antibodies (e.g., or ligelizumab) or antigen-binding fragment thereof, for use in, modifying the course of a disease or disorder disclosed herein, e.g. IgE driven allergic reaction to one or more allergens, e.g., anaphylaxis, in a subject in need thereof, comprising administering the subject a dose of about 24 mg to about 600 mg of an anti-IgE antibody (e.g., ligelizumab) or antigen-binding fragment thereof by subcutaneous injection.

The use of antibodies as the active ingredient of pharmaceuticals is now widespread, including the products HERCEPTIN™ (trastuzumab), RITUXAN™ (rituximab), SYNAGIS™ (palivizumab), etc. Techniques for purification of antibodies to a pharmaceutical grade are known in the art. When a therapeutically effective amount of an IgE antagonist, e.g., IgE binding molecules (e.g., IgE antibody or antigen-binding fragment thereof, e.g., ligelizumab) or IgE receptor binding molecules (e.g., IgE antibody or antigen-binding fragment thereof) is administered by intravenous, cutaneous or subcutaneous injection, the IgE antagonist will be in the form of a pyrogen-free, parenterally acceptable solution. A pharmaceutical composition for intravenous, cutaneous, or subcutaneous injection may contain, in addition to the IgE antagonist, an isotonic vehicle such as sodium chloride, Ringer's solution, dextrose, dextrose and sodium chloride, lactated Ringer's solution, or other vehicle as known in the art.

In practicing some of the methods of treatment or uses of the present disclosure, a therapeutically effective amount of an IgE antagonist, e.g., IgE binding molecule (e.g., IgE antibody or antigen-binding fragment thereof, e.g., ligelizumab) or IgE receptor binding molecule (e.g., IgE antibody or antigen-binding fragment thereof) is administered to a subject, e.g., a mammal (e.g., a human). While it is understood that the disclosed methods provide for treatment of food allergy subjects using an IgE antagonist (e.g., ligelizumab), this does not preclude that, if the subject is to be ultimately treated with an IgE antagonist, such IgE antagonist therapy is necessarily a monotherapy. Indeed, if a subject is selected for treatment with an IgE antagonist, then the IgE antagonist (e.g., ligelizumab) may be administered in accordance with the methods of the disclosure either alone or in combination with other agents and therapies for treating food allergy subjects, e.g., in combination with at least one additional food allergy agent. When co-administered with one or more additional food allergy agent(s), an IgE antagonist may be administered either simultaneously with the other agent, or sequentially. If administered sequentially, the attending physician will decide on the appropriate sequence of administering the IgE antagonist in combination with other agents and the appropriate dosages for co-delivery.

Disclosed herein are methods of, and anti-IgE antibodies (e.g., ligelizumab) or antigen-binding fragment thereof, for use in, modifying the course of a disease or disorder involving IgE, in a subject in need thereof, comprising administering the subject a dose of about 24 mg to about 600 mg of an anti-IgE antibody (e.g. ligelizumab) or antigen-binding fragment thereof by subcutaneous injection.

The anti-IgE antibody or antigen-binding fragment thereof (e.g., ligelizumab) may be administered to the patient intravenously (SC) every four weeks starting at week 0 and 4 and thereafter administered to the patient SC, e.g., at about 24 mg-about 360 mg (e.g., about 24 mg, about 240 mg) every four weeks, beginning during week 4. In this manner, the patient receives a SC dose during week 0, 4, 8, 12, 16, etc.

Alternatively the anti-IgE antibody or antigen-binding fragment thereof (e.g., ligelizumab) may be administered to the patient subcutaneously (s.c.) every four weeks starting at week 0 and thereafter administered to the patient s.c., e.g., at about 24 mg, about 72 mg, about 120 mg, about 240 mg, about 300 mg to about 360 mg (e.g., about 24 mg, about 240 mg) every four weeks, beginning during week 4. In this manner, the patient is dosed s.c. with about 24 mg, about 72 mg, about 120 mg to about 360 mg (e.g., about 24 mg, about 72 mg, about 120 mg, about 240 mg, about 300 mg to about 360 mg) of the anti-IgE antibody (e.g., ligelizumab) during weeks 0, 4, 8, 12, etc.

Preferably, the anti-IgE antibody or antigen-binding fragment thereof (e.g., ligelizumab) may be administered to the patient subcutaneously (s.c.) every four weeks starting at week 0 and thereafter administered to the patient s.c., e.g., at about 24 mg, about 72 mg, about 120 mg to about 360 mg (e.g., about 24 mg, about 72 mg, about 120 mg, about 240 mg, about 300 mg to about 360 mg) every four weeks, beginning during week 4. In this manner, the patient is dosed s.c. with about 24 mg, about 72 mg, about 120 mg to about 360 mg (e.g., about 24 mg, about 72 mg, about 120 mg about 240 mg, about 300 mg to about 360 mg) of the anti-IgE antibody (e.g., ligelizumab) during weeks 0, 4, 8, 12, 16, 20, etc.

More preferably, ligelizumab may be administered to the patient without a loading regimen, e.g., ligelizumab may be administered to the patient s.c. at about 24 mg, about 72 mg, about 120 mg to about 360 mg (e.g., about 24 mg, about 72 mg, about 120 mg, about 240 mg, about 300 mg to about 360 mg) every four weeks. In this manner, the patient is dosed s.c. with about 24 mg, about 72 mg, about 120 mg, about 240 mg, about 300 mg to about 360 mg (e.g., about 24 mg, about 72 mg, about 120 mg, about 240 mg, about 300 mg to about 360 mg) of ligelizumab during weeks 0, 4, 8, 12, etc.

More preferably, the anti-IgE antibody or antigen-binding fragment thereof (e.g., ligelizumab) may be administered to the patient subcutaneously (s.c.) every four weeks starting at week 0 and thereafter administered to the patient s.c., e.g., at about 240 mg to about 480 mg (e.g., about 240 mg, about 360 mg to about 480 mg) every four weeks, beginning during week 4. In this manner, the patient is dosed s.c. with about 240 mg to about 480 mg (e.g., about 240 mg, about 360 mg, or about 480 mg) of the anti-IgE antibody (e.g., ligelizumab) during weeks 0, 4, 8, 12, 16, 20, etc.

In some embodiments of the disclosed uses, methods, and kits, the subject has an IgE driven food allergy to one or more allergens is food allergy, e.g. peanuts allergy. In some embodiments, the subject may also have asthma and/or urticarial, e.g., has a disease or disorder selected from asthma, allergic asthma, rhinitis, allergic rhinitis, urticarial and CSU. In some embodiments of the disclosed uses, methods, and kits, the subject has, IgE driven food allergy to one or more allergens is food allergy, e.g. peanuts allergy, milk allergy or egg allergy. In other embodiments of the disclosed uses, methods, and kits, the subject has an IgE driven food allergy to one or more allergens is food allergy, e.g. peanuts allergy.

In some embodiments of the disclosed uses, methods, and kits, the anti-IgE antibody (e.g., ligelizumab) or antigen-binding fragment thereof, can be prescribed as first treatment or added on to any of the standard of care medications.

The timing of dosing is generally measured from the day of the first dose of anti-IgE antibody, e.g. ligelizumab, (which is also known as “baseline”). However, health care providers often use different naming conventions to identify dosing schedules, as shown in Table 1.

TABLE 1
Common naming conventions for dosing regimens. Bolded
items refer to the naming convention used herein.

| | | | | | | | |
|---|---|---|---|---|---|---|---|
| Week | 0/1 | 4/5 | 8/9 | 12/11 | 16/15 | 20/19 | etc. |
| 1^{st} day of week | 0/1 | 28/29 | 56/57 | 84/85 | 112/113 | 140/141 | etc. |

Notably, week zero may be referred to as week one by some health care providers, while day zero may be referred to as day one by some health care providers. Thus, it is possible that different physicians will designate, e.g., a dose as being given during week 4/on day 28, during week 4/on day 29, during week 4/on day 28, during week 4/on day 29, while referring to the same dosing schedule. For consistency, the first week of dosing will be referred to herein as week 0, while the first day of dosing will be referred to as day 1. However, it will be understood by a skilled artisan that this naming convention is simply used for consistency and should not be construed as limiting, i.e., weekly dosing is the provision of a weekly dose of the anti-IgE antibody regardless of whether the physician refers to a particular week as “week 0” or “week 1”.

In a one dosing regimen, the antibody is administered during week 0, 4, 8, 12, 16, 20, etc. Some providers may refer to this regimen as monthly dosing (or dosing every 4 weeks). It will be appreciated by a skilled artisan that administering a patient an injection at weeks 0 followed by once monthly dosing starting at week 4 is the same as: 1) administering the patient an injection at weeks 0 and 4, followed by once monthly dosing starting at week 8; 2) administering the patient an injection at weeks 0 and 4 followed by dosing every 4 weeks; and 3) administering the patient an injection at weeks 0 and 4 followed by monthly administration.

As used herein, the phrase “formulated at a dosage to allow [route of administration] delivery of [a designated dose]” is used to mean that a given pharmaceutical composition can be used to provide a desired dose of an anti-IgE antibody, e.g., ligelizumab, via a designated route of administration (e.g., s.c. or IV). As an example, if a desired s.c. dose is 240 mg, then a clinician may use 2 ml of an IgE antibody formulation having a concentration of 120 mg/ml, 1 ml of an anti-IgE antibody formulation having a concentration of 240 mg/ml, 0.5 ml of an anti-IgE antibody formulation having a concentration of 480 mg/ml, etc. In each such case, these anti-IgE antibody formulations are at a concentration high enough to allow subcutaneous delivery of the anti-IgE antibody. Subcutaneous delivery typically requires delivery of volumes of less than or equal to about 2 ml, preferably a volume of about 1 ml or less. Preferred formulations are ready-to-use liquid pharmaceutical compositions comprising about 24 mg/mL to about 120 mg/mL ligelizumab, in an aqueous solution containing L-histidine/L-histidine hydrochloride monohydrate as buffer agents, trehalose dehydrate as a stabilizer/tonicity adjuster, and polysorbate 20 as a surfactant.

As used herein, the phrase “container having a sufficient amount of the anti-IgE antibody to allow delivery of [a designated dose]” is used to mean that a given container (e.g., vial, pen, syringe) has disposed therein a volume of an anti-IgE antibody (e.g., as part of a pharmaceutical composition) that can be used to provide a desired dose. As an example, if a desired dose is 240 mg, then a clinician may use 2 mL from a container that contains an anti-IgE antibody formulation with a concentration of 120 mg/mL, 1 mL from a container that contains an anti-IgE antibody formulation with a concentration of 240 mg/mL, 0.5 mL from a container contains an anti-IgE antibody formulation with a concentration of 480 mg/ml, etc. In each such case, these containers have a sufficient amount of the anti-IgE antibody to allow delivery of the desired 240 mg dose.

In some embodiments of the disclosed uses, methods, and kits, the dose of the anti-IgE antibody (e.g., ligelizumab) or an antigen binding fragment thereof is about 240 mg, the anti-IgE antibody (e.g., ligelizumab) or an antigen binding fragment thereof is comprised in a liquid pharmaceutical formulation at a concentration of 120 mg/ml, and 2 ml of the pharmaceutical formulation is disposed within two pre-filled syringes (PFS), injection pens, or autoinjectors, each having 1 ml of the pharmaceutical formulation. In this case, the patient receives two injections of 1 ml each, for a total dose of 240 mg, during each administration. In some embodiments, the dose of the anti-IgE antibody (e.g., ligelizumab) is about 240 mg, the anti-IgE antibody (e.g., ligelizumab) is comprised in a liquid pharmaceutical formulation at a concentration of 120 mg/ml, and 2 ml of the pharmaceutical formulation is disposed within an autoinjector or PFS. In this case, the patient receives one injection of 2 ml, for a total dose of 240 mg, during each administration. In methods employing one injection of 2 ml (e.g., via a single PFS or autoinjector) (i.e., a “single-dose preparation”), the drug exposure (AUC) and maximal concentration (Cₘₐₓ) is equivalent (similar to, i.e., within the range of acceptable variation according to US FDA standards) to methods employing two injections of 1 ml (e.g., via two PFSs or two Als) (i.e., a “multiple-dose preparation”).

Disclosed herein are methods of treating food allergy (e.g., severe food allergy, comprising subcutaneously (s.c.) administering to a subject in need thereof a dose of about 24 mg-about 240 mg of an anti-IgE antibody (e.g., ligelizumab), weekly during week 0 and thereafter s.c. at a dose of about 24 mg-about 240 mg: a) monthly (every 4 weeks), beginning during week 4. Also disclosed herein is an anti-IgE antibody (e.g. ligelizumab), for use in treating food allergy, e.g., severe food allergy, comprising subcutaneously (s.c.) administering to a subject in need thereof a dose of about 24 mg-about 240 mg of the anti-IgE antibody, weekly during week 0 and thereafter s.c. at a dose of about 24 mg-about 240 mg monthly (every 4 weeks), beginning during week 4. Alternatively, disclosed herein is an anti-IgE antibody (e.g. ligelizumab), for use in the manufacture of a medicament for treating food allergy, comprising subcutaneously (s.c.) administering to a subject in need thereof a dose of about 24 mg-about 240 mg of the anti-IgE antibody, weekly during weeks 0 and thereafter s.c. at a dose of about 24 mg-about 240 mg monthly (every 4 weeks), beginning during week 4.

Disclosed herein are methods of treating food allergy, comprising subcutaneously (s.c.) administering to a patient in need thereof a dose of about 24 mg-about 240 mg of an anti-IgE antibody (e.g., ligelizumab), weekly during week 0 and thereafter s.c. at a dose of about 24 mg about 240 mg: a) monthly (every 4 weeks), beginning during week 4, wherein the anti-IgE antibody comprises an immunoglobulin V_{H} domain comprising the amino acid sequence set forth as SEQ ID NO:2 and an immunoglobulin V_{L} domain comprising the amino acid sequence set forth as SEQ ID NO:1.

Also disclosed herein is an anti-IgE antibody (e.g. ligelizumab), for use in treating food allergy, e.g., severe food allergy, comprising subcutaneously (s.c.) administering to a patient in need thereof a dose of about 24 mg-about 240 mg of the anti-IgE antibody, weekly during week 0 and thereafter s.c. at a dose of about 24 mg-about 240 mg monthly (every 4 weeks), beginning during week 4, wherein the anti-IgE antibody comprises an immunoglobulin VH domain comprising the amino acid sequence set forth as SEQ ID NO:2 and an immunoglobulin VL domain comprising the amino acid sequence set forth as SEQ ID NO:1.

Also disclosed herein is an anti-IgE antibody (e.g. ligelizumab), for use in treating food allergy, e.g., severe food allergy, comprising subcutaneously (s.c.) administering to a patient in need thereof a dose of about 24 mg-about 240 mg of the anti-IgE antibody, s.c. at a dose of about 24 mg-about 240 mg monthly (every 4 weeks), beginning during week 4, wherein the anti-IgE antibody comprises a variable light chain region comprising CDRL1, CDRL2, and CDRL3 and a variable heavy chain region comprising CDRH1, CDRH2, and CDRH3, wherein CDRL1 consists of SEQ ID NO:3, CDRL2 consists of SEQ ID NO:4, CDRL3 consists of SEQ ID NO:5, CDRH1 consists of SEQ ID NO:6, CDRH2 consists of SEQ ID NO:7, and CDRH3 consists of SEQ ID NO:8, wherein the antibody binds specifically to IgE.

Also disclosed herein is an anti-IgE antibody (e.g. ligelizumab), for use in treating food allergy, e.g., severe food allergy, comprising subcutaneously (s.c.) administering to a patient in need thereof a dose of about 360 mg of the anti-IgE antibody, s.c. at a dose of about 360 mg monthly (every 4 weeks), beginning during week 4, wherein the anti-IgE antibody comprises a variable light chain region comprising CDRL1, CDRL2, and CDRL3 and a variable heavy chain region comprising CDRH1, CDRH2, and CDRH3, wherein CDRL1 consists of SEQ ID NO:3, CDRL2 consists of SEQ ID NO:4, CDRL3 consists of SEQ ID NO:5, CDRH1 consists of SEQ ID NO:6, CDRH2 consists of SEQ ID NO:7, and CDRH3 consists of SEQ ID NO:8, wherein the antibody binds specifically to IgE.

In preferred embodiments of the disclosed methods, uses and kits, the dose of the anti-IgE antibody ligelizumab is about 120 mg or about 240 mg, about 360 mg, or about 480 mg.

In preferred embodiments of the disclosed methods, uses and kits, the anti-IgE antibody ligelizumab is administered s.c. at a dose of about 24 mg every four weeks.

In other preferred embodiments of the disclosed methods, uses and kits, the anti-IgE antibody ligelizumab is administered s.c. at a dose of about 72 mg every four weeks.

In other preferred embodiments of the disclosed methods, uses and kits, the anti-IgE antibody ligelizumab is administered s.c. at a dose of about 120 mg every four weeks.

In other preferred embodiments of the disclosed methods, uses and kits, the anti-IgE antibody ligelizumab is administered s.c. at a dose of about 240 mg every four weeks.

In other preferred embodiments of the disclosed methods, uses and kits, the anti-IgE antibody ligelizumab is administered s.c. at a dose of about 360 mg every four weeks.

In other preferred embodiments of the disclosed methods, uses and kits, the anti-IgE antibody ligelizumab is administered s.c. at a dose of about 480 mg every four weeks.

In other preferred embodiments of the disclosed methods, uses and kits, the anti-IgE antibody ligelizumab is administered s.c. at a dose of up to 12 mg/kg body weight/q4w.

In other preferred embodiments of the disclosed methods, uses and kits, the anti-IgE antibody ligelizumab for use in the prevention or reduction of IgE-mediated allergic reactions.

In other preferred embodiments of the disclosed methods, uses and kits, the anti-IgE antibody ligelizumab for use in the prevention or treatment of food allergy.

In other preferred embodiments of the disclosed methods, uses and kits, the anti-IgE antibody ligelizumab for use in the prevention or treatment of severe food allergy.

In other preferred embodiments of the disclosed methods, uses and kits, the anti-IgE antibody ligelizumab for use to prevent allergic reactions, including anaphylaxis, following accidental exposure to one or more food allergens in a subject in need thereof, wherein ligelizumab is administered s.c. at a dose of about 120 mg every four weeks.

In other preferred embodiments of the disclosed methods, uses and kits, the anti-IgE antibody ligelizumab for use to prevent allergic reactions, including anaphylaxis, following accidental exposure to one or more food allergens in a subject in need thereof, wherein ligelizumab is administered s.c. at a dose of about 240 mg every four weeks.

In other preferred embodiments of the disclosed methods, uses and kits, the anti-IgE antibody ligelizumab for use to prevent allergic reactions, including anaphylaxis, following accidental exposure to one or more food allergens in a subject in need thereof, wherein ligelizumab is administered s.c. at a dose of about 360 mg every four weeks.

In other preferred embodiments of the disclosed methods, uses and kits, the anti-IgE antibody ligelizumab for use to prevent allergic reactions, including anaphylaxis, following accidental exposure to one or more food allergens in a subject in need thereof, wherein ligelizumab is administered s.c. at a dose of about 480 mg every four weeks.

In other preferred embodiments of the disclosed methods, uses and kits, the anti-IgE antibody ligelizumab for use to prevent allergic reactions, including anaphylaxis, following accidental exposure to food allergens in conjunction with diet avoiding foods to which a subject is allergic, wherein ligelizumab is administered s.c. at a dose of about 120 mg every four weeks.

In other preferred embodiments of the disclosed methods, uses and kits, the anti-IgE antibody ligelizumab for use to prevent allergic reactions, including anaphylaxis, following accidental exposure to food allergens in conjunction with diet avoiding foods to which a subject is allergic, wherein ligelizumab is administered s.c. at a dose of about 240 mg every four weeks.

In other preferred embodiments of the disclosed methods, uses and kits, the anti-IgE antibody ligelizumab for use to prevent anaphylaxis following accidental exposure to food allergens in conjunction with diet avoiding foods to which a subject is allergic, wherein ligelizumab is administered s.c. at a dose of about 240 mg every four weeks.

In other preferred embodiments of the disclosed methods, uses and kits, the anti-IgE antibody ligelizumab for use to prevent anaphylaxis following accidental exposure to food allergens in conjunction with diet avoiding foods to which a subject is allergic, wherein ligelizumab is administered s.c. at a dose of about 360 mg every four weeks.

In other preferred embodiments of the disclosed methods, uses and kits, the anti-IgE antibody ligelizumab for use to prevent anaphylaxis following accidental exposure to one or more food allergens in conjunction with diet avoiding foods to which a subject is allergic, wherein ligelizumab is administered s.c. at a dose of about 120 mg every four weeks.

In other preferred embodiments of the disclosed methods, uses and kits, the anti-IgE antibody ligelizumab for use to prevent anaphylaxis following accidental exposure to one or more food allergens in a subject in need thereof, wherein ligelizumab is administered s.c. at a dose of about 240 mg every four weeks.

In other preferred embodiments of the disclosed methods, uses and kits, the anti-IgE antibody ligelizumab for use to prevent anaphylaxis following accidental exposure to one or more food allergens in a subject in need thereof, wherein ligelizumab is administered s.c. at a dose of about 360 mg every four weeks.

In other preferred embodiments of the disclosed methods, uses and kits, the anti-IgE antibody ligelizumab for use to prevent anaphylaxis following accidental exposure to one or more food allergens in a subject in need thereof, wherein ligelizumab is administered s.c. at a dose of about 480 mg every four weeks.

In other preferred embodiments of the disclosed methods, uses and kits, the anti-IgE antibody ligelizumab for use to prevent anaphylaxis following accidental exposure to one or more food allergens in a subject in need thereof, wherein ligelizumab is administered s.c. at a dose of about 120 mg every four weeks.

In other preferred embodiments of the disclosed methods, uses and kits, the anti-IgE antibody ligelizumab for use to prevent anaphylaxis following accidental exposure to one or more food allergens in a subject in need thereof, wherein ligelizumab is administered s.c. at a dose and a the dosing interval determined by serum total IgE level and body weight of said subject (measured before the start of treatment).

In other preferred embodiments of the disclosed methods, uses and kits, the anti-IgE antibody ligelizumab for use to prevent allergic reactions, including anaphylaxis, following accidental exposure to food allergens in conjunction with diet avoiding foods to which a subject is allergic, wherein ligelizumab is administered s.c. at a dose and a the dosing interval determined by serum total IgE level and body weight of said subject (measured before the start of treatment).

In other preferred embodiments of the disclosed methods, uses and kits, the anti-IgE antibody ligelizumab for use to prevent allergic reactions, including anaphylaxis, following accidental exposure to food allergens in conjunction with diet avoiding foods to which a subject is allergic, wherein ligelizumab is administered s.c. at a dose of about 240 mg every four weeks, and wherein said subject has no anaphylaxis or other allergic reactions to accidental exposure to food allergens during anti-IgE antibody treatment.

In other preferred embodiments of the disclosed methods, uses and kits, the anti-IgE antibody ligelizumab for use to prevent allergic reactions, including anaphylaxis, following accidental exposure to food allergens in conjunction with diet avoiding foods to which a subject is allergic, wherein ligelizumab is administered s.c. at a dose of about 120 mg every four weeks, and wherein said subject has no anaphylaxis or other allergic reactions to accidental exposure to food allergens during anti-IgE antibody treatment.

In preferred embodiments of the disclosed methods, uses and kits, prior to treatment with the anti-IgE antibody or antigen binding fragment, the subject has been previously treated with OIT.

In preferred embodiments of the disclosed methods, uses and kits, the anti-IgE antibody ligelizumab for use in combination with an allergen-specific oral immunotherapy to one or more allergens (oral immunotherapy OIT); e.g. single-allergen OIT or a multi-allergen OIT.

In preferred embodiments of the disclosed methods, uses and kits, the anti-IgE antibody ligelizumab for use in combination with an allergen-specific oral immunotherapy to one or more allergens (oral immunotherapy OIT); e.g. single-allergen OIT or a multi-allergen OIT, during the initial dose escalation (IDE) and maintenance phase of the OIT.

In some embodiments of the disclosed methods, uses and kits, the anti-IgE antibody is used as adjunct to OIT.

In some embodiments of the disclosed methods, uses and kits, the anti-IgE antibody reduces the frequency and/or severity of adverse events associated with the OIT.

In some embodiments of the disclosed methods, uses and kits, the anti-IgE antibody when administered as adjunct to OIT, the anti-IgE antibody shortens or eliminates the IDE phase in a subject.

In some embodiments of the disclosed methods, uses and kits, the anti-IgE antibody is administered as adjunct to OIT and the OIT is administered during the OIT maintenance dose, e.g. without the initial dose escalation (IDE).

In some embodiments of the disclosed methods, uses and kits, the anti-IgE antibody is administered as adjunct to an OIT, and the OIT is administered at its maintenance dose.

In some embodiments of the disclosed methods, uses and kits, the anti-IgE antibody is administered as adjunct to an OIT, and the OIT is administered at its maintenance dose, and wherein the anti-IgE antibody is ad mistered for a treatment duration of up to 6 months, e.g. of about 4 to about 6 months, of about 4 months, of about 3 months, or of about 2 months.

In some embodiments of the disclosed methods, uses and kits, the anti-IgE antibody induces sustained unresponsiveness/immunotolerance to food allergens in a subject.

In some embodiments of the disclosed methods, uses and kits, the anti-IgE antibody is administered in combination with an OIT.

In some embodiments of the disclosed methods, uses and kits, the anti-IgE antibody is administered in combination with an OIT and the anti-IgE antibody induces sustained unresponsiveness/immunotolerance to food allergens in a subject.

In some embodiments of the disclosed methods, uses and kits, the anti-IgE antibody is administered in combination with an OIT, and wherein the anti-IgE antibody is administered for a treatment duration of up to 3 years, e.g. of about 2 to about 3 years, of about 3 years, or of about 2 years.

In some embodiments of the disclosed methods, uses and kits, prior to treatment with the anti-IgE antibody, the patient has not been previously treated with OIT.

In preferred embodiments of the disclosed methods, uses and kits, the dose of the anti-IgE antibody is about 24 mg. In other preferred embodiments of the disclosed methods, uses and kits, the dose of the anti-IgE antibody is about 72 mg. In other preferred embodiments of the disclosed methods, uses and kits, the dose of the anti-IgE antibody is about 120 mg. In other preferred embodiments of the disclosed methods, uses and kits, the dose of the anti-IgE antibody is about 240 mg. In other preferred embodiments of the disclosed methods, uses and kits, the dose of the anti-IgE antibody is about 300 mg. In other preferred embodiments of the disclosed methods, uses and kits, the dose of the anti-IgE antibody is about 360 mg. In other preferred embodiments of the disclosed methods, uses and kits, the dose of the anti-IgE antibody is about 480 mg.

In preferred embodiments of the disclosed methods, uses and kits, the subject has food allergy, e.g., severe food allergy.

In preferred embodiments of the disclosed methods, uses and kits, the subject is an adult. In some embodiments of the disclosed methods, uses and kits, the subject is an adolescent. In some embodiments of the disclosed methods, uses and kits, the subject is a pediatric subject.

In preferred embodiments of the disclosed methods, uses and kits, the anti-IgE antibody is disposed in a pharmaceutical formulation, wherein said pharmaceutical formulation further comprises a buffer and a stabilizer. In some embodiments of the disclosed methods, uses and kits, the pharmaceutical formulation is in liquid form (ready-to-use). In some embodiments of the disclosed methods, uses and kits, the pharmaceutical formulation is in lyophilized form. In some embodiments of the disclosed methods, uses and kits, pharmaceutical formulation is disposed within pre-filled syringes, vials, injection pens, or autoinjectors.

In preferred embodiments of the disclosed methods, uses and kits, the dose of the anti-IgE antibody about 24 mg, 72 mg, 120 mg, 240 mg, 300 mg, 360 mg, or 480 mg in the pharmaceutical formulation is disposed within means for administering selected from the group consisting of a pre-filled syringe, an injection pen, and an autoinjector, and said means is disposed within a kit, and the kit further comprises instructions for use.

In preferred embodiments of the disclosed methods, uses and kits, the dose of the anti-IgE antibody is about 120 mg, the pharmaceutical formulation is disposed within an autoinjector or a pre-filled syringe, and the autoinjector or pre-filled syringe is disposed within a kit, and the kit further comprises instructions for use.

In preferred embodiments of the disclosed methods, uses and kits, the dose of the anti-IgE antibody is about 240 mg, the pharmaceutical formulation is disposed within autoinjectors or pre-filled syringes, the autoinjectors or pre-filled syringes are disposed within a kit, and the kit further comprises instructions for use.

In preferred embodiments of the disclosed methods, uses and kits, the dose is 240 mg, which is administered as a single subcutaneous administration in a total volume of 2 ml from a formulation comprising 120 mg/ml of the anti-IgE antibody or antigen binding fragment, wherein the pharmacological exposure of the patient to the anti-IgE antibody is equivalent to the pharmacological exposure of the patient to the anti-IgE antibody using two separate subcutaneous administrations of a total volume of 1 ml each of the same formulation.

In preferred embodiments of the disclosed methods, uses and kits, the dose is 240 mg, which is administered as two separate subcutaneous administrations in a volume of 1 ml each from a formulation comprising 120 mg/ml of the anti-IgE antibody, e.g. ligelizumab.

In preferred embodiments of the disclosed methods, uses and kits, the dose is 300 mg, which is administered as two separate subcutaneous administrations in a volume of 1 ml each from a formulation comprising 150 mg/ml of the anti-IgE antibody, e.g. ligelizumab.

In preferred embodiments of the disclosed methods, uses and kits, the dose is 360 mg, which is administered as three separate subcutaneous administrations in a volume of 1 ml each from a formulation comprising 120 mg/ml of the anti-IgE antibody, e.g. ligelizumab.

In preferred embodiments of the disclosed methods, uses and kits, the dose is 480 mg, which is administered as four separate subcutaneous administrations in a volume of 1 ml each from a formulation comprising 120 mg/ml of the anti-IgE antibody, e.g. ligelizumab.

In preferred embodiments of the disclosed methods, uses and kits, the pharmaceutical formulation of the anti-IgE antibody is disposed within autoinjectors or pre-filled syringes, and the autoinjectors or pre-filled syringes are suitable for home administration by a subject in need thereof.

In preferred embodiments of the disclosed methods, uses and kits, the subject tolerates a highest dose of at least 600-mg of protein from a relevant allergen or allergens with no more than mild symptoms at a double-blind placebo-controlled food challenge (DBPCFC), after treatment with the anti-IgE antibody.

In preferred embodiments of the disclosed methods, uses and kits, the subject tolerates a highest dose of at least 1000-mg of protein from a relevant allergen or allergens with no more than mild symptoms at a double-blind placebo-controlled food challenge (DBPCFC), after treatment with the anti-IgE antibody.

In preferred embodiments of the disclosed methods, uses and kits, the subject tolerates a highest dose of at least 3000-mg of protein from a relevant allergen or allergens with no more than mild symptoms at a double-blind placebo-controlled food challenge (DBPCFC), after treatment with the anti-IgE antibody.

In preferred embodiments of the disclosed methods, uses and kits, the subject achieves not having any objective allergic reaction to the double-blind, placebo controlled, oral food challenge (DBPCFC) at levels of 600 mg (1044 mg cumulative), 1,000 mg (2044 mg cumulative) or 3,000 mg (5044 mg cumulative) of peanut protein after week 12 of treatment with ligelizumab as disclosed herein, e.g. 120 mg or 240 mg or 360 mg or 480 mg, s.c., administered every 4 weeks.

In preferred embodiments of the disclosed methods, uses and kits, may result in at least an amelioration of one or more symptoms associated with the allergy, e.g., as described above in the introduction section. Allergy symptoms that may be ameliorated, but are not limited to: eczema, asthma, atopic dermatitis, bronchospasm, cough, rhinorrhea, angioedema, gastric hypermotility, urticaria (hives), pruritis, fatigue, bradycardia, and/or hypotension. The magnitude of the symptom reduction may vary, where in some instances the magnitude is 2-fold or greater, e.g., 5-fold or greater, including 10-fold or greater, e.g., as compared to a suitable control. In some instances, treatment of an allergy results the subject being cured of the allergy, such that the subject no longer suffers from the allergy. In some embodiments of allergy treatment methods, the methods include administering to a subject am anti-IgE antibody, such as described above.

In preferred embodiments of the disclosure, the anti-IgE antibody is a monoclonal antibody.

In preferred embodiments of the disclosure, the anti-IgE antibody is a human or humanized antibody.

In preferred embodiments of the disclosure, the anti-IgE antibody is a human antibody.

In preferred embodiments of the disclosed methods, uses and kits, the anti-IgE antibody is a human monoclonal antibody.

In preferred embodiments of the disclosed methods, uses and kits, the anti-IgE antibody has a Tₘₐₓ of about 2-14 days.

In preferred embodiments of the disclosed methods, uses and kits, the anti-IgE antibody has an absolute bioavailability of about 47-100%.

In some embodiments, the anti-IgE antibody, such as ligelizumab, may refer to antibodies which have demonstrated to be biosimilar to or interchangeable to ligelizumab. Those antibodies may be administered according the embodiments which refer to ligelizumab administration, as herein disclosed.

### Kits

The disclosure also encompasses kits for treating particular subjects having disease or disorder involving IgE. Such kits comprise an anti-IgE antibody (e.g., ligelizumab) or antigen-binding fragment thereof, (e.g., in liquid or lyophilized form) or a pharmaceutical composition comprising the anti-IgE antibody (described supra). Additionally, such kits may comprise means for administering the anti-IgE antibody or antigen-binding fragment thereof (e.g., an auto-injector, a syringe and vial, a prefilled syringe, a prefilled pen) and instructions for use. These kits may contain additional therapeutic agents (described supra) for treating the disease or disorder involving IgE, e.g., for delivery in combination with the enclosed anti-IgE antibody or antigen-binding fragment thereof, e.g., ligelizumab. Such kits may also comprise instructions for administration of the anti-IgE antibody or antigen-binding fragment thereof, (e.g., ligelizumab.) to treat the subject. Such instructions may provide the dose (e.g., 24 mg, 72 mg, 75 mg, 120 mg, 240 mg, 360 mg or 480 mg), route of administration (e.g., IV, s.c.), and dosing regimen (e.g., every two or four weeks, during e.g. 12 to 16 weeks) for use with the enclosed anti-IgE antibody or antigen-binding fragment thereof, e.g., ligelizumab.

The phrase “means for administering” is used to indicate any available implement for systemically administering a drug to a subject, including, but not limited to, a pre-filled syringe, a vial and syringe, an injection pen, an auto-injector, an IV drip and bag, a pump, etc. With such items, a subject may self-administer the drug (i.e., administer the drug without the assistance of a physician) or a medical practitioner may administer the drug.

Disclosed herein are kits for use in modifying the disease course in a subject having disease or disorder involving IgE, comprising an anti-IgE antibody (e.g., ligelizumab) or antigen-binding fragment thereof. In some embodiments, the kit further comprises means for administering the anti-IgE antibody (e.g., ligelizumab) or antigen-binding fragment thereof, to the subject.

### General

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure.

These examples should in no way be construed as limiting the scope of the disclosed subject matter, as defined by the appended claims.

### EXAMPLES

### Example 1. Clinical Study with Omalizumab as Monotherapy and as Adjunct Therapy to Multi-Allergen Oral Immunotherapy (OIT) in Food Allergic Children and Adults

This is a multi-center, randomized, double-blind, placebo-controlled study in participants 2 to less than 56 years of age who are allergic to peanut and at least two other foods (including milk, egg, wheat, cashew, hazelnut, or walnut).

Omalizumab is supplied in pre-filled syringes (PFS). PFS of omalizumab will be provided to the clinical research units as 75 mg and 150 mg dosage forms.

Eligible participants are randomized to receive omalizumab by subcutaneous injection either every 2 weeks or every 4 weeks for 16 to 20 weeks. The dose administered and the dosing interval are determined by serum total IgE level and body weight (measured before the start of treatment).

After completion of eight weeks of open label omalizumab, participants will be randomized 1:1 to either:
Omalizumab-facilitated oral immunotherapy (OIT): Open label omalizumab+Multi-allergen OIT for eight weeks, followed by placebo for omalizumab+Multi-allergen OIT for 44 weeks OR
Omalizumab+placebo OIT: Open label omalizumab+placebo for Multi-allergen OIT for eight weeks, followed by omalizumab+placebo for Multi-allergen OIT for 44 weeks.

Multi-allergen OIT will be any of the following drug products: peanut, milk, egg, wheat, cashew, hazelnut, and walnut (all food protein flours). A prescription for each participant for the appropriate dose of each of the allergens will be prepared.

The study will randomize 225 participants. Maximum individual study participation will be up to 84 weeks for treatment.

### Example 2. Dose of Ligelizumab

The following ligelizumab doses have been tested in the clinical programs: 12 mg, 24 mg, 36 mg, 72 mg, 180 mg and 240 mg SC q4w, 280 mg SC q2w and 120 mg SC single dose. Overall, no apparent dose-dependent safety signals (except for a trend in injection site reactions, which can be easily managed clinically) have been observed to date.

Simulations with a model describing the activity of the FcεRI (wheal diameter of a skin prick test and PC15=the dose of allergen required to trigger an acute 15% decrease of FEV1) and selected critical biomarkers required for its activation (density of basophil-bound FcεRI and its occupancy with IgE) were performed in order to identify suitable doses supportive of a treatment for food allergies using ligelizumab. The simulations demonstrated that PD biomarkers of basophil reactivity (FcεRI density and its occupancy with IgE) are critical in the pathophysiology of food allergies and can discriminate across doses beyond more distal clinical PD outcomes. A complete level of suppression of the IgE/FcεRI pathway throughout the four week dosing interval is a fundamental therapeutic target to ensure maximal protection against potentially life-threatening allergic reactions in food allergic patients. These simulations showed that the 120 mg/q4w and regimen 240 mg/q4w are able maximize the clinical outcome.

### Example 3. Clinical Study Milk and Egg Allergy

A randomized, double-blind, placebo controlled, 12 week basket study assessing two regimens (120 mg and 240 mg administered monthly) of ligelizumab s.c. in patients 6 to 55 year old with demonstrated milk or egg allergies. Approximately 240 patients are randomized without any pre-defined baseline stratification by age sub-groups. Half of the patients will are randomized into the milk sub-study and the other half into the egg sub-study.

The basket study assessing milk and egg allergens is evaluating the efficacy of ligelizumab 240 mg and 120 mg (SCq4w, i.e. subcutaneous injection every 4 weeks), compared to placebo, in the proportion of patients who do not exhibit an objective allergic reaction to the DBPCFC at multiple levels of 1′000 mg (no cumulative) and 3′000 mg (no cumulative) of milk/egg proteins at week 12.

### Example 4. Clinical Study Peanut Allergy

A multi-center, randomized, double-blind and placebo-controlled study to assess the safety and clinical efficacy of two dosing regimens of ligelizumab (240 mg and 120 mg) SCq4w (subcutaneous injection every 4 weeks) in participants with a medically confirmed diagnosis of IgE-mediated peanut allergy

This “peanut study” is evaluating the efficacy of ligelizumab 240 mg and 120 mg (SCq4w), compared to placebo, in the proportion of patients who do not exhibit an objective allergic reaction to the double-blind, placebo controlled, oral food challenge (DBPCFC) at levels of 600 mg (1044 mg cumulative), 1′000 mg (2044 mg cumulative) and 3′000 mg (5044 mg cumulative) of peanut protein at week 12.

This is a 52-week, Phase 3 multi-center, randomized, double-blind and placebo-controlled study to assess the safety and clinical efficacy of two dosing regimens of ligelizumab (240 mg and 120 mg) SCq4w (subcutaneous injection every 4 weeks) in participants with a medically confirmed diagnosis of IgE-mediated peanut allergy. Approximately 486 participants are randomized to ligelizumab 240 mg, ligelizumab 120 mg, or placebo (5 treatment arms, randomization ratio of 2:2:2:2:1) for the double-blind placebo-controlled treatment period (up to Week 12). Participants initially assigned to the 8-week placebo arms will receive the first dose of blinded ligelizumab treatment at the Week 8 visit. Participants initially assigned to the 16-week placebo arm receive the last dose of placebo before the DBPCFC at week 12 and the first dose of blinded ligelizumab treatment at the Week 16 visit.

Participants will be stratified based on region, total IgE at baseline (<350 IU/mL; 2350 IU/mL) and age (6-11y, 12-17y, and 18-55y). Approximately the same number of participants will be randomized into each age group.

Age groups are defined as follows:
6-11y corresponds to ≥6 to <12 years of age
12-17y corresponds to ≥12 to <18 years of age
18-55y corresponds to ≥18 to ≤55 years of age

The choice of peanut as the main food allergen in this study relates to the following key factors:
It represents an important unmet medical need as food allergic reactions are most often severe with this allergen, and is a leading cause of fatal and near-fatal anaphylaxis in the US
Most patients (>80%) retain their phenotype into adulthood which enables a study across multiple age groups

The two selected dosing regimens (120 mg SCq4w and 240 mg SCq4V) reflect the clinical goal to maximize the protection against potentially life-threatening allergic reactions triggered by accidental exposure to food allergens. In fact the cascade of events leading to full blown anaphylaxis is mediated by the cross-linking of the high affinity IgE receptor (FcεRI) on effector cells (basophils and mast cells) that triggers the release of the inflammatory mediators. In this pathophysiologic context, a profound blockade of IgE binding to the FcεRI receptors with the consequent maximal downregulation of these FcεRIs is required because data suggests that basophils can respond maximally to stimulation with only 5000 antigen-specific IgE molecules per cell. Maximal suppression is also desirable considering that the IgE system is exposed to external factors like infections that may further increase its reactivity.

The proposed two doses have been selected based on simulations with a model build on atopic healthy volunteers and asthmatic participants then adapted in another version with CSU data for a sensitivity analysis (QGE031 simulations food allergy, Novartis). The activity of the FcεRI (wheal diameter of a skin prick test and PC15=the dose of allergen required to trigger an acute 15% decrease of FEV1) and selected critical biomarkers required for its activation provides the output from this model. These simulations show that the 120 mg SCq4w regimen might suffice to maximize the clinical outcome for most of participants. More proximal biomarkers of basophil reactivity discriminate the two doses by showing that maximal suppression would be reached for more participants with the 240 mg SCq4w dosing regimen. A dose above 240 mg SCq4w does not provide additional suppression of these biomarkers.

The decision to include the 120 mg on top of the 240 mg regimen is based on two main considerations:
The study population will be characterized by a range of baseline total IgE levels and body weights. These parameters are well known to impact the exposure of ligelizumab and some participants with lower IgE and body weight might sufficiently benefit of the 120 mg SC4qW regimen.

A lower dosing regimen of 120 mg SCq4w is also important to generate a broad range of data (exposure/response), supporting robust modeling at the end of the Phase 3 program to support the final posology for registration purposes.

### Key Inclusion Criteria

Male or female participants who are ≥6 and ≤55 years of age at the time of signing informed consent/assent.
Documented medical history of allergy to peanuts or peanut-containing foods.
Positive peanut-specific immunoglobulin E (peanut sIgE), ≥6 kUA/L at Screening visit 1 (Screening 1).
Positive skin prick test (SPT) for peanut allergen at Screening 1 defined as an average diameter (Longest diameter and mid-point orthogonal diameter) a 4 mm wheal compared to saline control.
A positive peanut DBPCFC at baseline (Screening Visit 2, Part 1 and Part 2 DBPCFC) defined as the occurrence of dose-limiting symptoms at a single dose ≤100 mg of peanut protein, and no occurrence on placebo. Eligibility to proceed with the DBPCFC requires fulfillment of all other eligibility criteria.
Participants must weigh ≥20 kg at Screening 1.

### Key Exclusion Criteria

Total IgE >2000 IU/mL at Screening 1.
History of severe or life-threatening hypersensitivity event needing an ICU admission or intubation within 60 days prior to baseline DBPCFC (Screening visit 2).
Participants with uncontrolled asthma (according to GINA guidelines, GINA 2020) who meet any of the following criteria:
   FEV1<80% of subject's predicted normal value at Screening visit 1
One hospitalization for asthma within 12 months prior to Screening visit 1

Rescue medication: Any treatment deemed necessary by the investigator can be used to treat adverse events, including allergic reactions. Typically, this includes epinephrine, SABA, anti-histamines and saline bolus.

The choice of peanut as the main food allergen in this study relates to the following key factors:
It represents an important unmet medical need as food allergic reactions are most often severe with this allergen, and is a leading cause of fatal and near-fatal anaphylaxis in the US
Most patients (>80%) retain their phenotype into adulthood which enables a study across multiple age groups.

### Example 5. Food Allergies Pediatric Study (6m-6y Old Children)

This study is a 52-week, double blind, placebo controlled study in children ≥6m to ≤6y of age with a clinically demonstrated IgE-driven food allergy against at least one of the following foods: peanut, milk, or egg. It assesses the efficacy and safety of at least one regimen of ligelizumab over one year: ligelizumab (240 mg and 120 mg) SCq4w (subcutaneous injection every 4 weeks).

### Example 6. Hazelnut Allergy Study

This is a 24-weeks, double blind, placebo-controlled study to assess the efficacy and safety of ligelizumab in participants ≥6y to ≤55y with hazelnut allergy. The study will demonstrate further the benefit of ligelizumab across all food allergies (“allergic agnostic”).

The objective of this Phase 3b study is to evaluate the safety and clinical efficacy of ligelizumab 120 mg and 240 mg SCq4w to ensure protection against food allergic reactions by decreasing the sensitivity to oral hazelnut allergen in participants aged ≥6 to ≤55 years with hazelnut allergy, compared to placebo. It evaluates the safety and tolerability of ligelizumab, compared to placebo, as measured by the proportion of participants who can tolerate a single dose of hazelnut protein without DLS during the DBPCFC at Week 24 at different thresholds; with the primary objective defined a single dose of ≥600 mg (1044 mg cumulative tolerated dose) of hazelnut protein.

Key population features:
Subjects must be aged between 6-55 years at the time of screening
Subject must have a medically documented clinical history of allergy to hazelnut or hazelnut-containing foods
Subjects must have specific immune response to hazelnut immunoglobulin E (sigE), ≥6 kUA/L at Screening 1
Subjects must have a positive skin prick test (SPT) with hazelnut extract at baseline defined as an average diameter 24 mm wheal compared to saline control
Subjects must have a positive hazelnut DBPCFC at baseline (Screening 2, Part 1 and 2 OCF) defined as the occurrence of DLS at a single dose ≤100 mg of hazelnut protein and no occurrence on placebo. Eligibility to proceed with the DBPCFC requires fulfillment of all other eligibility criteria.

### Example 7. High Dose Ligelizumab Study

This study is a Phase 3 multi-center and open-label extension study to evaluate the long-term efficacy and safety of ligelizumab 120 mg, 240 mg, and 360 mg SCq4w in participants with a confirmed diagnosis of food allergy.

Responder Status defines three levels of response based on the outcomes of OFC:

| Responder Status (RS) | Peanut | Milk/Egg/Hazelnut |
|---|---|---|
| Maximum responder [MR] | No DLS up to max dose (3000 | No DLS up to max dose (3000 |
| | mg) | mg) |
| Responder [R] | Maximum tolerated dose | Maximum tolerated dose |
| | of ≥600 mg and <3000 mg | of ≥1000 mg and <3000 mg |
| Non-responder [NR] | Maximum tolerated dose | Maximum tolerated dose |
| | of ≤300 mg | of ≤600 mg |

DLS = Dose-limiting symptom

A non-responder (NR) status reflects both the presence of the phenotype and an insufficient effect of the investigational drug based on the given regimen. An up-titration (max 360 mg in adults) or a prolonged exposure (one additional year at the same level if up-titration is not feasible) are the recommended strategies.

A responder (R) status reflects the presence of the phenotype and a clinically meaningful treatment benefit for the patient. In this case the protocol recommends either up-titration (where feasible) or prolonged treatment with the same regimen.

A maximum responder (MR) status might reflect either an optimal effect of the investigational drug or a change in the underlying phenotype (loss of disease either following natural history or through potential disease modification of the investigational treatment). In this case, the protocol might recommend a wash out to assess whether further treatment is still required. In case of sustained un-responsiveness (over a year), the patient will remain in the study without treatment (“extended wash out”) to keep monitoring his condition through selected biomarkers. In case of resurgence of the phenotype, patients will be brought back to the treatment regimen that led to MR.

### Example 8. Study Assessing Ligelizumab in Patients with Recurrent Spontaneous Anaphylaxis Due to Unknown and/or Unavoidable Triggers (Prevention of IgE-Mediated Anaphylaxis)

The randomized, placebo controlled study, assessing 240 mg-360 mg of ligelizumab SC q4w, study includes patients suffering from severe/serious allergic (IgE-mediated) reactions triggered by additional allergens (e.g. insect stings venom and drugs) beyond food. In this group of patients we will also include those suffering from events of unidentified triggers (idiopathic).

Patients with recurrent spontaneous anaphylaxis due to unknown and/or unavoidable triggers with at least three (or at least two or at least one) events in the 12 months prior to Visit 1 are recruited.
Primary endpoint: annualized rate of anaphylactic events (confirmed by adjudication)
Other selected endpoints:
   Rate of overall hypersensitivity reactions (including severity)
   Health Care utilization
   QoL
   Safety & tolerability

The data from the clinical trials described herein support the finding that IgE suppression by ligelizumab is a therapeutic approach that is allergen-agnostic.

What is claimed is:

1-5. (canceled)

6. A method of treating, preventing or reducing an IgE mediated allergic reaction in a subject in need thereof, comprising administering a therapeutically effective amount of an anti-IgE antibody or antigen binding fragment.

7. The method according to claim 6, wherein the IgE mediated allergic reaction is triggered by consumption of, or exposure to a food item.

8. The method according to claim 6, wherein the IgE mediated allergic reaction is triggered by exposure to a non-food allergen.

9. The method according to claim 6, wherein the IgE mediated allergic reaction is anaphylaxis.

10. The method according to claim 6, wherein the anti-IgE antibody is ligelizumab.

11. The method according to claim 10, wherein ligelizumab is administered at a dose of about 120 mg to about 600 mg, e.g. at a maximum dose of 600 mg.

12. The method according to claim 11, wherein ligelizumab is administered at a dose of about 240 mg.

13. The method according to claim 11, wherein ligelizumab is administered at a dose of about 360 mg.

14. The method according to claim 11, wherein ligelizumab is administered at a dose of about 480 mg.

15. The method according to claim 6, wherein the anaphylaxis is idiopathic anaphylaxis.

16. The method according to claim 6, wherein the patient has a confirmed diagnosis of IgE mediated food allergy to one or more allergens.

17. The method according to claim 6, wherein the patient suffers from a food allergy, wherein the food allergy is a peanuts allergy, a milk allergy, a egg allergy or a hazelnut allergy.

18. The method according to any one of claims 10 to 17, wherein ligelizumab is administered every two to four weeks.
